(19) United States
(12) Patent Application Publication
(10) Pub. No.: US 2011/0086762 A1
(43) Pub. Date: Apr. 14, 2011

(54) 4'4'-Dioxaspiro-Spirocyclically Substituted Tetramates

(75) Inventors: Reiner Fischer, Monheim (DE); Rolf Pontzen, Leichlingen (DE); Ronald Vermeer, Monheim (DE); Eva-Maria Franken, Limonest (DE); Olga Malsam, Rosrath (DE); Leonardo Pitta, Leverkusen (DE); Udo Reckmann, Koln (DE)

(73) Assignee: Bayer CropScience AG, Monheim (DE)

(21) Appl. No.: 12/933,040

(22) PCT Filed: Mar. 16, 2009
(86) PCT No.: PCT/EP09/01897

§ 371 Date: Dec. 7, 2010

(30) Foreign Application Priority Data
Mar. 19, 2008 (EP) 08153002.4

(51) Int. Cl. A01N 43/36; C07D 491/107; A01P 15/00; A01P 21/00

(52) U.S. Cl. 504/283; 548/408; 514/409

(57) ABSTRACT

The present invention relates to novel 4′4′-dioxaspiro-spirocyclically substituted tetramates of the formula (I)

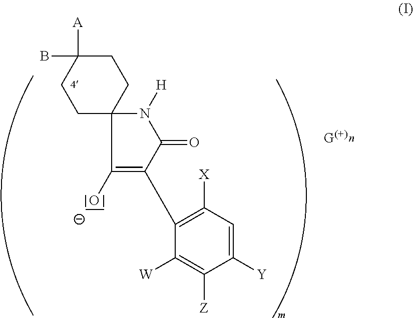

in which A, B, G, m, n, W, X, Y and Z
to a plurality of processes for their preparation and to their use as pesticides and/or herbicides. The invention also provides selectively herbicidal compositions comprising firstly the 4′4′-dioxaspiro-spirocyclically substituted tetramates and secondly a crop plant compatibility-improving compound.

The present invention also relates to novel water-soluble concentrates of 4′4′-dioxaspiro-spirocyclically substituted tetramates and their enols, to processes for preparing these formulations and to their use as pesticides and/or herbicides.

The present invention furthermore relates to increasing the activity of crop protection compositions comprising in particular 4′4′-dioxaspiro-spirocyclically substituted tetramates by adding ammonium salts or phosphonium salts and, if appropriate, penetrants, to the corresponding compositions, to processes for their preparation and to their use in crop protection as insecticides and/or acaricides and/or for preventing unwanted plant growth.

The present invention relates to novel 4′4′-dioxaspiro-spirocyclically substituted tetramates, to a plurality of processes for their preparation and to their use as pesticides and/or herbicides. The invention also provides selectively herbicidal compositions comprising, firstly, the 4′4′-dioxaspiro-spirocyclically substituted tetramates and, secondly, a crop plant compatibility-improving compound.

The present invention also relates to novel water-soluble concentrates (SL formulations) of 4′4′-dioxaspiro-spirocyclically substituted tetramates and their enols, to processes for preparing these formulations and to their use as pesticides and/or herbicides.

The present invention furthermore relates to the boosting of the action of crop protection compositions comprising, in particular, 4′4′-dioxaspiro-spirocyclically substituted tetramates, through the addition of ammonium salts or phosphonium salts and optionally penetrants, to the corresponding compositions, to processes for producing them and to their application in crop protection as insecticides and/or acaricides and/or for preventing unwanted plant growth.

For 3-acylpyrrolidine-2,4-diones pharmaceutical properties have been previously described (S. Suzuki et al. Chem. Pharm. Bull. 15 1120 (1967)). Furthermore, N-phenylpyrrolidine-2,4-diones have been synthesized by R. Schmierer and H. Mildenberger (Liebigs Ann. Chem. 1985, 1095). Biological activity of these compounds has not been described.

EP-A-0 262 399 and GB-A-2 266 888 disclose similarly structured compounds (3-arylpyrrolidine-2,4-diones) for which, however, no herbicidal, insecticidal or acaricidal action has been disclosed. Known compounds with herbidical, insecticidal or acaricidal action are unsubstituted bicyclic 3-arylpyrrolidine-2,4-dione derivatives (EP-A-355 599, EP-A-415 211 and JP-A-12-053 670) and also substituted monocyclic 3-arylpyrrolidine-2,4-dione derivatives (EP-A-377 893 and EP-A-442 077).

Additionally known are polycyclic 3-arylpyrrolidine-2,4-dione derivatives (EP-A-442 073) and 1H-arylpyrrolidinedione derivatives (EP-A-456 063, EP-A-521 334, EP-A-596 298, EP-A-613 884, EP-A-613 885, WO 95/01 997, WO 95/26 954, WO 95/20 572, EP-A-0 668 267, WO 96/25 395, WO 96/35 664, WO 97/01 535, WO 97/02 243, WO 97/36 868, WO 97/43275, WO 98/05638, WO 98/06721, WO 98/25928, WO 99/24437, WO 99/43649, WO 99/48869 and WO 99/55673, WO 01/17972, WO 01/23354, WO 01/74770, WO 03/013249, WO 03/062244, WO 2004/007448, WO 2004/024 688, WO 04/065366, WO 04/080962, WO 04/111042, WO 05/044791, WO 05/044796, WO 05/048710, WO 05/049569, WO 05/066125, WO 05/092897, WO 06/000355, WO 06/029799, WO 06/056281, WO 06/056282, WO 06/089633, WO 07/048545, DE-A-102005059892, WO 07/073856, WO 07/096058, WO 07/121868, WO 07/140881, WO 08/067873, WO 08/067910, WO 08/067911, WO 08/138551, PCT/EP2008/005973 and EP 07117104). Furthermore known are ketal-substituted 1-H-arylpyrrolidine-2,4-diones from WO 99/16748 and (spiro)-ketal-substituted N-alkoxyalkoxy-substituted arylpyrrolidinediones from JP-A-14 205 984 and Ito M. et. al., Bioscience, Biotechnology and Biochemistry 67, 1230-1238, (2003). The addition of safeners to ketoenols is also known in principle from WO 03/013249. Moreover, WO 06/024411 and PCT/EP2008/005185 disclose herbicidal compositions comprising ketoenols.

However, the herbicidal and/or acaricidal and/or insecticidal activity and/or activity spectrum and/or the plant compatibility of the known compounds, in particular with respect to crop plants, is/are not always satisfactory.

The present invention now provides novel compounds of the formula (I)

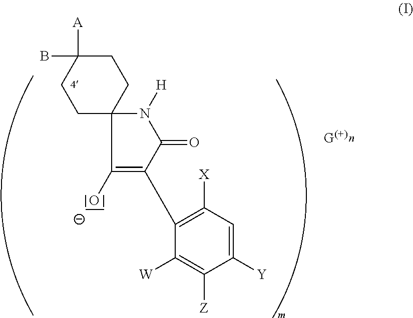

in which
W represents hydrogen, alkyl, alkenyl, alkynyl, halogen, alkoxy, haloalkyl, haloalkoxy or cyano,
X represents halogen, alkyl, alkenyl, alkynyl, alkoxy, haloalkyl, haloalkoxy, nitro or cyano,
Y and Z independently of one another represent hydrogen, alkyl, alkenyl, alkynyl, alkoxy, halogen, haloalkyl, haloalkoxy, cyano or nitro,
A and B and the carbon atom to which they are attached represent a five- to seven-membered ketal which is in each case optionally substituted by alkyl, haloalkyl, alkoxy, alkoxyalkyl or optionally substituted phenyl,
G represents a metal ion equivalent or ammonium ion,
m represents the number 1 or 2,
n represents the number 1 or 2.

Depending inter alia on the nature of the substituents, the compounds of the formula (I) may be present as geometrical and/or optical isomers or isomer mixtures of varying composition which, if appropriate, may be separated in a customary manner. The present invention provides the pure isomers and the tautomer and isomer mixtures, their preparation and use and materials comprising them. However, for the sake of simplicity, hereinbelow only compounds of the formula (I) are referred to, although what is meant are both the pure compounds and, if appropriate, mixtures having various proportions of isomeric and tautomeric compounds.

Furthermore, it has been found that the novel compounds of the formula (I) are obtained by one of the processes described below:
(A) substituted 4′4′-dioxaspiro-spirocyclically substituted tetramates of the formula (I)

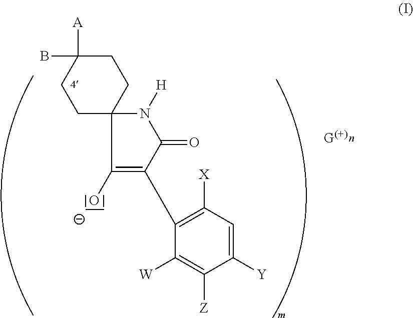

in which
A, D, m, n, W, X, Y and Z have the meanings given above,
are obtained when
N-acylamino acid esters of the formula (II)

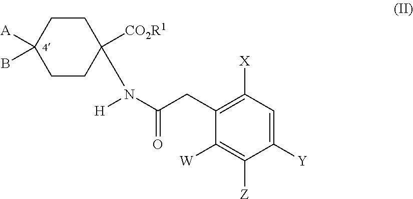

in which
A, B, W, X, Y and Z have the meanings given above,
and
R¹ represents alkyl (preferably C₁-C₆-alkyl),
are condensed intramolecularly in the presence of a diluent and in the presence of a metal base
(B) Furthermore, it has been found that compounds of the formula (I) shown above in which A, B, G, m, n, W, X, Y and Z have the meanings given above are obtained when compounds of the formula (I′),

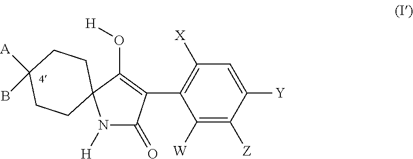

in which A, B, W, X, Y and Z have the meanings given above, are in each case reacted
α) with metal compounds of the formula (III) or (IV)
   G(OR²)ₙ (III), G(H)ₙ (IV)
in which
G represents a mono- or divalent metal (preferably an alkali metal or alkaline earth metal such as lithium, sodium, potassium, caesium, magnesium or calcium),
n represents the number 1 or 2 and
R² represents hydrogen or alkyl (preferably C₁-C₈-alkyl),
if appropriate in the presence of a diluent
or
β) with amines of the formula (V) or ammonium compounds of the formula (VI)

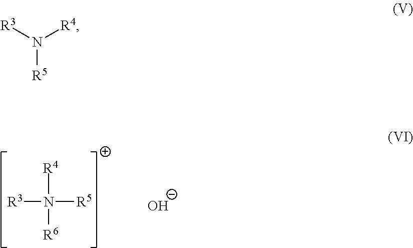

in which
R³, R⁴, R⁵, R⁶ independently of one another represent hydrogen, C₁-C₈ alkyl, C₁-C₄-alkoxy-C₂-C₄-alkyl, poly-(C₁-C₄-alkoxy)-C₂-C₄-alkenyl, C₃-C₈-alkenyl, C₁-C₈-alkoxy or optionally halogen-, alkyl- or alkoxy-substituted benzyl,
if appropriate in the presence of a diluent.

Furthermore, it has been found that the novel compounds of the formula (I) are very effective as pesticides, preferably as insecticides, acaricides and/or herbicides.

Surprisingly, it has now also been found that 4′4′-dioxaspiro-spirocyclically substituted tetramates, when used together with the crop plant compatibility-improving compounds (safeners/antidotes) described below, efficiently prevent damage to the crop plants and can be used in a particularly advantageous manner as broad-spectrum combination preparations for the selective control of unwanted plants in crops of useful plants, such as, for example, in cereals, but also in maize, soya beans and rice.

The invention also provides selective herbicidal materials comprising an effective amount of an active compound combination comprising, as components,
(a′) at least one 4′4′-dioxaspiro-spirocyclically substituted tetramate of the formula (I) in which A, B, G, m, n, W, X, Y and Z have the meaning given above
   and
(b) at least one crop plant compatibility-improving compound from the following group of compounds:
   4-dichloroacetyl-1-oxa-4-azaspiro[4.5]decane (AD-67, MON-4660), 1-dichloroacetylhexahydro-3,3,8a-trimethylpyrrolo[1,2-a]pyrimidin-6(2H)-one (dicyclonon, BAS-145138), 4-dichloroacetyl-3,4-dihydro-3-methyl-2H-1,4-benzoxazine (benoxacor), 1-methylhexyl 5-chloroquinoline-8-oxyacetate (cloquintocet-mexyl—cf. also related compounds in EP-A-86750, EP-A-94349, EP-A-191736, EP-A-492366), 3-(2-chlorobenzyl)-1-(1-methyl-1-phenylethyl)urea (cumyluron), α-(cyanomethoximino)phenylacetonitrile (cyometrinil), 2,4-dichlorophenoxyacetic acid (2,4-D), 4-(2,4-dichlorophenoxy)butyric acid (2,4-DB), 1-(1-methyl-1-phenylethyl)-3-(4-methylphenyl)urea (daimuron, dymron), 3,6-dichloro-2-methoxybenzoic acid (dicamba), S-1-methyl-1-phenylethyl piperidine-1-thiocarboxylate (dimepiperate), 2,2-dichloro-N-(2-oxo-2-(2-propenylamino)ethyl)-N-(2-propenyl)acetamide (DKA-24), 2,2-dichloro-N,N-di-2-propenylacetamide (dichlormid), 4,6-dichloro-2-phenylpyrimidine (fenclorim), ethyl 1-(2,4-dichlorophenyl)-5-trichloromethyl-1H-1,2,4-triazole-3-carboxylate (fenchlorazole-ethyl—cf. also related compounds in EP-A-174562 and EP-A-346620), phenylmethyl 2-chloro-4-trifluoromethylthiazole-5-carboxylate (flurazole), 4-chloro-N-(1,3-dioxolan-2-ylmethoxy)-α-trifluoroacetophenone oxime (fluxofenim), 3-dichloroacetyl-5-(2-furanyl)-2,2-dimethyloxazolidine (furilazole, MON-13900), ethyl 4,5-dihydro-5,5-diphenyl-3-isoxazolecarboxylate (isoxadifen-ethyl—cf. also related compounds in WO-A-95/07897), 1-(ethoxycarbonyl)ethyl 3,6-dichloro-2-methoxybenzoate (lactidichlor), (4-chloro-o-tolyloxy)acetic acid (MCPA), 2-(4-chloro-o-tolyloxy)propionic acid (mecoprop), diethyl 1-(2,4-dichlorophenyl)-4,5-dihydro-5-methyl-1H-pyrazole-3,5-dicarboxylate (mefenpyr-diethyl—cf. also related compounds in WO-A-91/07874), 2-dichloromethyl-2-methyl-1,3-dioxolane (MG-191), 2-propenyl-1-oxa-4-azaspiro[4.5]decane-4-carbodithioate (MG-838), 1,8-naphthalic anhydride, α-(1,3-dioxolan-2-ylmethoximino)phenylacetonitrile (oxabetrinil), 2,2-dichloro-N-(1,3-dioxolan-2-ylmethyl)-N-(2-propenyl)acetamide (PPG-1292), 3-dichloroacetyl-2,2-dimethyloxazolidine (R-28725), 3-dichloroacetyl-2,2,5-trimethyloxazolidine (R-29148), 4-(4-chloro-o-tolyl)butyric acid, 4-(4-chlorophenoxy)butyric acid, diphenylmethoxyacetic acid, methyl diphenylmethoxyacetate, ethyl diphenylmethoxyacetate, methyl 1-(2-chlorophenyl)-5-phenyl-1H-pyrazole-3-carboxylate, ethyl 1-(2,4-dichlorophenyl)-5-methyl-1H-pyrazole-3-carboxylate, ethyl 1-(2,4-dichlorophenyl)-5-isopropyl-1H-pyrazole-3-carboxylate, ethyl 1-(2,4-dichlorophenyl)-5-(1,1-dimethylethyl)-1H-pyrazole-3-carboxylate, ethyl 1-(2,4-dichlorophenyl)-5-phenyl-1H-pyrazole-3-carboxylate (cf. also related compounds in EP-A-269806 and EP-A-333131), ethyl 5-(2,4-dichlorobenzyl)-2-isoxazoline-3-carboxylate, ethyl 5-phenyl-2-isoxazoline-3-carboxylate, ethyl 5-(4-fluorophenyl)-5-phenyl-2-isoxazoline-3-carboxylate (cf. also related compounds in WO-A-91/08202), 1,3-dimethylbut-1-yl 5-chloroquinoline-8-oxyacetate, 4-allyloxybutyl 5-chloroquinoline-8-oxyacetate, 1-allyloxyprop-2-yl 5-chloroquinoline-8-oxyacetate, methyl 5-chloroquinoxaline-8-oxyacetate, ethyl 5-chloroquinoline-8-oxyacetate, allyl 5-chloroquinoxaline-8-oxyacetate, 2-oxoprop-1-yl 5-chloroquinoline-8-oxyacetate, diethyl 5-chloroquinoline-8-oxymalonate, diallyl 5-chloroquinoxaline-8-oxymalonate, diethyl 5-chloroquinoline-8-oxymalonate (cf. also related compounds in EP-A-582198), 4-carboxychroman-4-ylacetic acid (AC-304415, cf. EP-A-613618), 4-chlorophenoxyacetic acid, 3,3′-dimethyl-4-methoxybenzophenone, 1-bromo-4-chloromethylsulphonylbenzene, 1-[4-(N-2-methoxybenzoylsulphamoyl)phenyl]-3-methylurea (also known as N-(2-methoxybenzoyl)-4-[(methylaminocarbonyl)amino]benzenesulphonamide), 1-[4-(N-2-methoxybenzoylsulphamoyl)-phenyl]-3,3-dimethylurea, 1-[4-(N-4,5-dimethylbenzoylsulphamoyl)phenyl]-3-methylurea, 1-[4-(N-naphthylsulphamoyl)phenyl]-3,3-dimethylurea, N-(2-methoxy-5-methylbenzoyl)-4-(cyclopropylaminocarbonyl)benzenesulphonamide,
   and/or one of the following compounds, defined by general formulae, of the general formula (IIa)

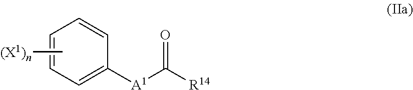

or of the general formula (IIb)

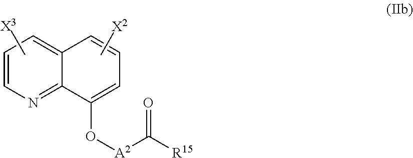

or of the formula (IIc)

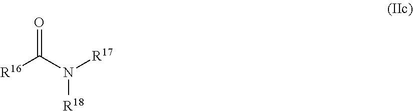

where
n represents a number from 0 to 5,
A¹ represents one of the divalent heterocyclic groupings shown below,

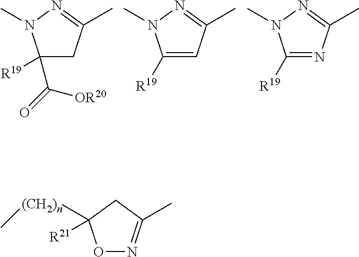

n represents a number from 0 to 5,
A² represents optionally C₁-C₄-alkyl- and/or C₁-C₄-alkoxycarbonyl-substituted alkanediyl having 1 or 2 carbon atoms,
R¹⁴ represents hydroxyl, mercapto, amino, C₁-C₆-alkoxy, C₁-C₆-alkylamino or di(C₁-C₄-alkyl)amino,
R¹⁵ represents hydroxyl, mercapto, amino, C₁-C₆-alkoxy, C₁-C₆-alkylthio, C₁-C₆-alkylamino or di(C₁-C₄-alkyl)amino,
R¹⁶ represents in each case optionally fluorine-, chlorine- and/or bromine-substituted C₁-C₄-alkyl,
R¹⁷ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-alkyl-substituted phenyl,
R¹⁸ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-alkyl-substituted phenyl, or together with R¹⁷ represents C₃-C₆-alkanediyl or C₂-C₅-oxaalkanediyl, each of which is optionally substituted by C₁-C₄-alkyl, phenyl, furyl, a fused benzene ring or by two substituents which, together with the C atom to which they are attached, form a 5- or 6-membered carbocycle,
R¹⁹ represents hydrogen, cyano, halogen, or represents in each case optionally fluorine-, chlorine- and/or bromine-substituted C₁-C₄-alkyl, C₃-C₆-cycloalkyl or phenyl,
R²⁰ represents hydrogen, in each case optionally hydroxyl-, cyano-, halogen- or C₁-C₄-alkoxy-substituted C₁-C₆-alkyl, C₃-C₆-cycloalkyl or tri(C₁-C₄-alkyl)silyl,
R²¹ represents hydrogen, cyano, halogen, or represents in each case optionally fluorine-, chlorine- and/or bromine-substituted C₁-C₄-alkyl, C₃-C₆-cycloalkyl or phenyl,
X¹ represents nitro, cyano, halogen, C₁-C₄-alkyl, C₁-C₄-haloalkyl, C₁-C₄-alkoxy or C₁-C₄-haloalkoxy,
X² represents hydrogen, cyano, nitro, halogen, C₁-C₄-alkyl, C₁-C₄-haloalkyl, C₁-C₄-alkoxy or C₁-C₄-haloalkoxy,
X³ represents hydrogen, cyano, nitro, halogen, C₁-C₄-alkyl, C₁-C₄-haloalkyl, C₁-C₄-alkoxy or C₁-C₄-haloalkoxy,
   and/or the following compounds, defined by general formulae
   of the general formula (IId)

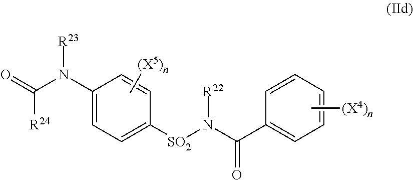

or of the general formula (IIe)

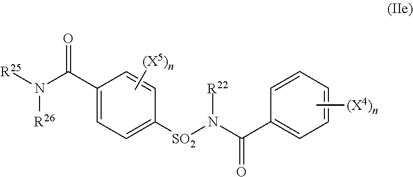

where
n represents a number from 0 to 5,
R²² represents hydrogen or C₁-C₄-alkyl,
R²³ represents hydrogen or C₁-C₄-alkyl,
R²⁴ represents hydrogen, in each case optionally cyano-, halogen- or C₁-C₄-alkoxy-substituted C₁-C₆-alkyl, C₁-C₆-alkoxy, C₁-C₆-alkylthio, C₁-C₆-alkylamino or di(C₁-C₄-alkyl)amino, or in each case optionally cyano-, halogen- or C₁-C₄-alkyl-substituted C₃-C₆-cycloalkyl, C₃-C₆-cycloalkyloxy, C₃-C₆-cycloalkylthio or C₃-C₆-cycloalkylamino,
R²⁵ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, or optionally cyano-, halogen- or C₁-C₄-alkyl-substituted C₃-C₆-cycloalkyl,
R²⁶ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, optionally cyano-, halogen- or C₁-C₄-alkyl-substituted C₃-C₆-cycloalkyl, or optionally nitro-, cyano-, halogen-, C₁-C₄-alkyl-, C₁-C₄-alkoxy- or C₁-C₄-haloalkoxy-substituted phenyl, or together with R²⁵ represents in each case optionally C₁-C₄-alkyl-substituted C₂-C₆-alkanediyl or C₂-C₅-oxaalkanediyl,
X⁴ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy, and
X⁵ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy.

Furthermore, it has been found that compositions comprising a phase comprising at least one dissolved active compound of the formula (I) or (I′)

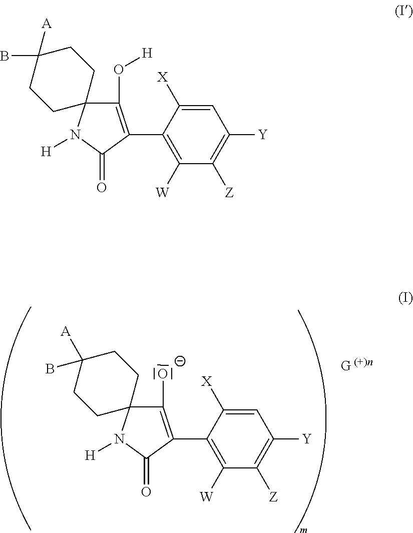

in which

A, B, G, m, n, W, X, Y and Z have the meaning given above

on application following dilution at pH values of ≧5 to suitable concentrations have a more rapid onset of action and/or better compatibility with crop plants and/or higher activity than, for example, corresponding SC formulations.

This is particularly surprising since, owing to the relatively high solubility in water of the active compounds of the formula (I′) at pH values of ≧5, starting from any formulation type following dilution with water concentrations are reached which are significantly lower than the expected solubility of the active compound in water—the activity thus being independent of the original formulation type. Of course, the solubility kinetics are also a relevant parameter in the preparation of the spray liquor. However, the person skilled in the art would expect a formulation which, in addition to the active compound, also comprises higher concentrations of surfactants, to have higher solubility kinetics. Surprisingly, this has not been found.

In the formulations according to the invention, the active compounds of the formula (I) are in dissolved form even in the concentrated compositions.

Accordingly, the present invention provides compositions comprising at least one solvent and at least one compound of the formula (I) or (I′) in dissolved form.

The present invention also provides processes for preparing water-soluble concentrates comprising at least one compound of the formula (I) or (I′).

Moreover, the present invention provides the use of compositions comprising a phase comprising at least one compound of the formula (I) or (I′) in dissolved form for controlling unwanted plant growth and/or animal pests.

In general, the compounds of the formula (I) or (I′) listed above can be used according to the invention.

Compounds of the formula (I) which are preferred according to the invention are the compounds mentioned in the Preparation Examples, where each compound comprised therein is preferred per se.

Compounds of the formula (I′) which are preferred according to the invention are listed in Table A, where each compound comprised therein is preferred per se.

TABLE A
(I′)

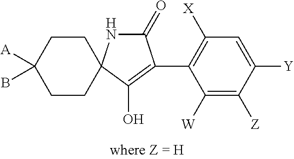

| | | | | | | |
|---|---|---|---|---|---|---|
| | | | | | | Known from |
| | | | | | | WO 06/089633; |
| Ex. No. | W | X | Y | A | B | Ex. No. |

| | | | | | |
|---|---|---|---|---|---|
| I′-1 | CH₃ | CH₃ | CH₃ | —O—(CH₂)₂—O— | I-1-a-2 |
| I′-2 | CH₃ | CH₃ | Cl | —O—(CH₂)₂—O— | I-1-a-4 |
| I′-3 | CH₃ | CH₃ | Br | —O—(CH₂)₂—O— | I-1-a-26 |
| I′-4 | CH₃ | CH₃ | CH₃ | —O—(CH₂)₃—O— | I-1-a-18 |
| I′-5 | CH₃ | CH₃ | Cl | —O—(CH₂)₃—O— | I-1-a-14 |
| I′-6 | CH₃ | CH₃ | Br | —O—(CH₂)₃—O— | I-1-a-19 |

The formula (I) provides a general definition of the compounds according to the invention. Preferred substituents or ranges of the radicals given in the formulae mentioned above and below are illustrated below:
W preferably represents hydrogen, chlorine, bromine, C₁-C₄-alkyl, C₁-C₄-alkoxy, C₁-C₂-haloalkyl or C₁-C₂-haloalkoxy,
X preferably represents chlorine, bromine, C₁-C₄-alkyl, C₁-C₄-alkoxy, C₁-C₄-haloalkyl, C₁-C₄-haloalkoxy or cyano,
Y and Z independently of one another preferably represent hydrogen, fluorine, chlorine, bromine, C₁-C₄-alkyl, C₁-C₆-alkoxy, C₁-C₄-haloalkyl, C₁-C₄-haloalkoxy or cyano,
A and B and the carbon atom to which they are attached preferably represent a five- or six-membered ketal which is optionally mono- to trisubstituted by C₁-C₄-alkyl, C₁-C₃-haloalkyl, C₁-C₄-alkoxy or C₁-C₄-alkoxy-C₁-C₂-alkyl,
G preferably represents lithium, sodium, potassium, caesium, magnesium-halogen cations, magnesium, calcium or an ammonium ion

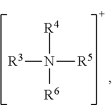

in which R³, R⁴, R⁵, R⁶ have the meaning mentioned above,
m preferably represents the number 1 or 2,
n preferably represents the number 1 or 2.

In the radical definitions mentioned as being particularly preferred, halogen represents fluorine, chlorine and bromine, in particular fluorine and chlorine.
W particularly preferably represents hydrogen, chlorine, bromine, methyl, ethyl, methoxy, ethoxy or trifluoromethyl,
X particularly preferably represents chlorine, bromine, methyl, ethyl, methoxy, ethoxy, trifluoromethyl, difluoromethoxy, trifluoromethoxy or cyano,
Y and Z independently of one another particularly preferably represent hydrogen, fluorine, chlorine, bromine, methyl, ethyl, methoxy, trifluoromethyl, trifluoromethoxy or cyano,
A and B and the carbon atom to which they are attached particularly preferably represent a five- or six-membered ketal which is optionally mono- or disubstituted by methyl, ethyl, propyl, trifluoromethyl, monochloromethyl, methoxy, ethoxy, methoxymethyl or ethoxymethyl,
G particularly preferably represents lithium, sodium, potassium, caesium, a magnesium chloride cation, a magnesium bromide cation, a magnesium iodide cation, magnesium, calcium or an ammonium ion

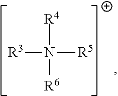

in which R³, R⁴, R⁵, R⁶ independently of one another particularly preferably represent hydrogen, C₁-C₆-alkyl or benzyl.
m particularly preferably represents the number 1 or 2,
n particularly preferably represents the number 1 or 2.
W very particularly preferably represents hydrogen, chlorine, bromine, methyl, ethyl or methoxy, (in particular methyl),
X very particularly preferably represents chlorine, bromine, methyl, ethyl, methoxy or ethoxy, (in particular methyl)
Y and Z independently of one another very particularly preferably represent hydrogen, chlorine, bromine or methyl, (Y represents in particular methyl or chlorine, Z represents in particular hydrogen),
A and B and the carbon atom to which they are attached very particularly preferably represent a five- or six-membered ketal which is optionally mono- or disubstituted by methyl, ethyl, propyl, monochloromethyl or methoxymethyl, (in particular —O—(CH₂)₂—O—),
G very particularly preferably represents lithium, sodium, potassium, caesium, a magnesium bromide cation, magnesium, calcium or an ammonium ion

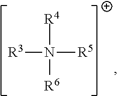

in which R³, R⁴, R⁵, R⁶ independently of one another very particularly preferably represent C₁-C₄-alkyl or benzyl, (G represents in particular lithium, sodium, potassium, magnesium or calcium),
m very particularly preferably represents the number 1 or 2,
n very particularly preferably represents the number 1 or 2.

The general or preferred radical definitions or illustrations listed above can be combined with one another as desired, i.e. including combinations between the respective ranges and preferred ranges.

Preference according to the invention is given to the compounds of the formula (I) which contain a combination of the meanings listed above as being preferred (preferable).

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

Special preference according to the invention is given to the compounds of the formula (I) which contain a combination of the meanings listed above as being especially preferred.

Saturated or unsaturated hydrocarbon radicals, such as alkyl, alkanediyl or alkenyl, can in each case be straight-chain or branched as far as this is possible, including in combination with heteroatoms, such as, for example, in alkoxy.

Unless indicated otherwise, optionally substituted radicals may be mono- or polysubstituted, where in the case of polysubstitutions the substituents may be identical or different.

In addition to the compounds mentioned in the Preparation Examples, the following compounds of the formula (I) may be specifically mentioned:

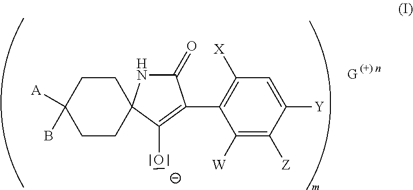

TABLE 1
G⁽⁺⁾ⁿ = Na⁺; m = 1; Z = H

| | | | | |
|---|---|---|---|---|
| W | X | Y | A | B |

| | | | |
|---|---|---|---|
| CH₃ | CH₃ | CH₃ | —O—(CH₂)₂—O— |
| CH₃ | CH₃ | Cl | —O—(CH₂)₂—O— |
| CH₃ | CH₃ | Br | —O—(CH₂)₂—O— |
| CH₃ | CH₃ | CH₃ | —O—(CH₂)₃—O— |
| CH₃ | CH₃ | Cl | —O—(CH₂)₃—O— |
| CH₃ | CH₃ | Br | —O—(CH₂)₃—O— |

TABLE 2
A, B, W, X and Y as stated in Table 1

G⁽⁺⁾ⁿ = K⁺; m = 1

TABLE 3
A, B, W, X and Y as stated in Table 1

G⁽⁺⁾ⁿ = Li⁺; m = 1

TABLE 4

A, B, W, X and Y as stated in Table 1
G⁽⁺⁾ₙ = Mg²⁺; m = 2

TABLE 5

A, B, W, X and Y as stated in Table 1
G⁽⁺⁾ₙ = Ca²⁺; m = 2

TABLE 6

A, B, W, X and Y as stated in Table 1
G⁽⁺⁾ₙ = [H₂N(CH₃)₂]⁺; m = 1

Preferred meanings of the groups listed above in connection with the crop plant compatibility-improving compounds (“herbicide safeners”) of the formulae (IIa), (IIb), (IIc), (IId) and (IIe) are defined below.
n preferably represents the number 0, 1, 2, 3 or 4.
A¹ preferably represents one of the divalent heterocyclic groupings shown below

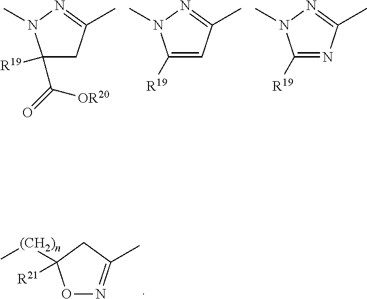

A² preferably represents in each case optionally methyl-, ethyl-, methoxycarbonyl- or ethoxycarbonyl-substituted methylene or ethylene,
R¹⁴ preferably represents hydroxyl, mercapto, amino, methoxy, ethoxy, n- or i-propoxy, n-, i-, s- or t-butoxy, methylthio, ethylthio, n- or i-propylthio, n-, s- or t-butylthio, methylamino, ethylamino, n- or i-propylamino, n-, s- or t-butylamino, dimethylamino or diethylamino.
R¹⁵ preferably represents hydroxyl, mercapto, amino, methoxy, ethoxy, n- or i-propoxy, n-, s- or t-butoxy, methylthio, ethylthio, n- or i-propylthio, n-, s- or t-butylthio, methylamino, ethylamino, n- or i-propylamino, n-, s- or t-butylamino, dimethylamino or diethylamino.
R¹⁶ preferably represents in each case optionally fluorine-, chlorine- and/or bromine-substituted methyl, ethyl, n- or i-propyl.
R¹⁷ preferably represents hydrogen, in each case optionally fluorine- and/or chlorine-substituted methyl, ethyl, n- or i-propyl, n-, s- or t-butyl, propenyl, butenyl, propynyl or butynyl, methoxymethyl, ethoxymethyl, methoxyethyl, ethoxyethyl, dioxolanylmethyl, furyl, furyl-methyl, thienyl, thiazolyl, piperidinyl, or optionally fluorine-, chlorine-, methyl-, ethyl-, n- or i-propyl-, n-, s- or t-butyl-substituted phenyl.
R¹⁸ preferably represents hydrogen, in each case optionally fluorine- and/or chlorine-substituted methyl, ethyl, n- or i-propyl, n-, i-, s- or t-butyl, propenyl, butenyl, propynyl or butynyl, methoxymethyl, ethoxymethyl, methoxyethyl, ethoxyethyl, dioxolanylmethyl, furyl, furyl-methyl, thienyl, thiazolyl, piperidinyl, or optionally fluorine-, chlorine-, methyl-, ethyl-, n- or i-propyl-, n-, s- or t-butyl-substituted phenyl, or together with R¹⁷ represents one of the radicals —CH₂—O—CH₂—CH₂— and —CH₂—CH₂—O—CH₂—CH₂— which are optionally substituted by methyl, ethyl, furyl, phenyl, a fused benzene ring or by two substituents which, together with the C atom to which they are attached, form a 5- or 6-membered carbocycle.
R¹⁹ preferably represents hydrogen, cyano, fluorine, chlorine, bromine, or represents in each case optionally fluorine-, chlorine- and/or bromine-substituted methyl, ethyl, n- or i-propyl, cyclopropyl, cyclobutyl, cyclopentyl, cyclohexyl or phenyl.
R²⁰ preferably represents hydrogen, in each case optionally hydroxyl-, cyano-, fluorine-, chlorine-, methoxy-, ethoxy-, n- or i-propoxy-substituted methyl, ethyl, n- or i-propyl, n-, s- or t-butyl.
R²¹ preferably represents hydrogen, cyano, fluorine, chlorine, bromine, or represents in each case optionally fluorine-, chlorine- and/or bromine-substituted methyl, ethyl, n- or i-propyl, n-, s- or t-butyl, cyclopropyl, cyclobutyl, cyclopentyl, cyclohexyl or phenyl.
X¹ preferably represents nitro, cyano, fluorine, chlorine, bromine, methyl, ethyl, n- or i-propyl, n-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy.
X² preferably represents hydrogen, nitro, cyano, fluorine, chlorine, bromine, methyl, ethyl, n- or i-propyl, n-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy.
X³ preferably represents hydrogen, nitro, cyano, fluorine, chlorine, bromine, methyl, ethyl, n- or i-propyl, n-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy.
R²² preferably represents hydrogen, methyl, ethyl, n- or i-propyl.
R²³ preferably represents hydrogen, methyl, ethyl, n- or i-propyl.
R²⁴ preferably represents hydrogen, in each case optionally cyano-, fluorine-, chlorine-, methoxy-, ethoxy-, n- or i-propoxy-substituted methyl, ethyl, n- or i-propyl, n-, s- or t-butyl, methoxy, ethoxy, n- or i-propoxy, n-, s- or t-butoxy, methylthio, ethylthio, n- or i-propylthio, n-, s- or t-butylthio, methylamino, ethylamino, n- or i-propylamino, n-, i-, s- or t-butylamino, dimethylamino or diethylamino, or in each case optionally cyano-, fluorine-, chlorine-, bromine-, methyl-, ethyl-, n- or i-propyl-substituted cyclopropyl, cyclobutyl, cyclopentyl, cyclohexyl, cyclopropyloxy, cyclobutyloxy, cyclopentyloxy, cyclohexyloxy, cyclopropylthio, cyclobutylthio, cyclopentylthio, cyclohexylthio, cyclopropylamino, cyclobutylamino, cyclopentylamino or cyclohexylamino.
R²⁵ preferably represents hydrogen, in each case optionally cyano-, hydroxyl-, fluorine-, chlorine-, methoxy-, ethoxy-, n- or i-propoxy-substituted methyl, ethyl, n- or i-propyl, n-, i- or s-butyl, in each case optionally cyano-, fluorine-, chlorine- or bromine-substituted propenyl, butenyl, propynyl or butynyl, or in each case optionally cyano-, fluorine-, chlorine-, bromine-, methyl-, ethyl-, n- or i-propyl-substituted cyclopropyl, cyclobutyl, cyclopentyl or cyclohexyl.
R²⁶ preferably represents hydrogen, in each case optionally cyano-, hydroxyl-, fluorine-, chlorine-, methoxy-, ethoxy-, n- or i-propoxy-substituted methyl, ethyl, n- or i-propyl, n-, i- or s-butyl, in each case optionally cyano-, fluorine-, chlorine- or bromine-substituted propenyl, butenyl, propynyl or butynyl, in each case optionally cyano-, fluorine-, chlorine-, bromine-, methyl-, ethyl-, n- or i-propyl-substituted cyclopropyl, cyclobutyl, cyclopentyl or cyclohexyl, or optionally nitro-, cyano-, fluorine-, chlorine-, bromine-, methyl-, ethyl-, n- or i-propyl-, n-, s- or t-butyl-, trifluoromethyl-, methoxy-, ethoxy-, n- or i-propoxy-, difluoromethoxy- or trifluoromethoxy-substituted phenyl, or together with R²⁵ represents in each case optionally methyl- or ethyl-substituted butane-1,4-diyl(trimethylene), pentane-1,5-diyl, 1-oxabutane-1,4-diyl or 3-oxapentane-1,5-diyl
X⁴ preferably represents nitro, cyano, carboxyl, carbamoyl, formyl, sulphamoyl, hydroxyl, amino, fluorine, chlorine, bromine, methyl, ethyl, n- or i-propyl, n-, s- or t-butyl, trifluoromethyl, methoxy, ethoxy, n- or i-propoxy, difluoromethoxy or trifluoromethoxy.
X⁵ preferably represents nitro, cyano, carboxyl, carbamoyl, formyl, sulphamoyl, hydroxyl, amino, fluorine, chlorine, bromine, methyl, ethyl, n- or i-propyl, n-, s- or t-butyl, trifluoromethyl, methoxy, ethoxy, n- or i-propoxy, difluoromethoxy or trifluoromethoxy.

Examples of the compounds of the formula (IIa) which are very particularly preferred as herbicide safeners according to the invention are listed in the table below.

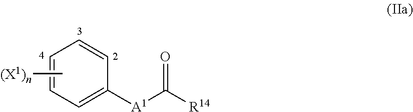

TABLE
Examples of compounds of the formula (IIa)

| Example | (Positions) | | |
|---|---|---|---|
| No. | (X¹)ₙ | A¹ | R¹⁴ |
| IIa-1 | (2) Cl, (4) Cl | 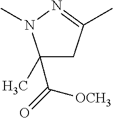 | OCH₃ |
| IIa-2 | (2) Cl, (4) Cl | 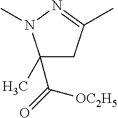 | OCH₃ |
| IIa-3 | (2) Cl, (4) Cl | 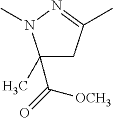 | OC₂H₅ |
| IIa-4 | (2) Cl, (4) Cl | 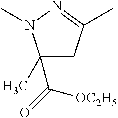 | OC₂H₅ |
| IIa-5 | (2) Cl | 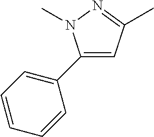 | OCH₃ |
| IIa-6 | (2) Cl, (4) Cl | 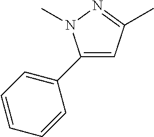 | OCH₃ |
| IIa-7 | (2) F | 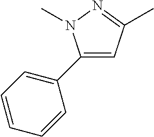 | OCH₃ |
| IIa-8 | (2) F | 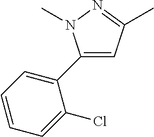 | OCH₃ |
| IIa-9 | (2) Cl, (4) Cl | 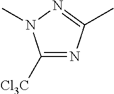 | OC₂H₅ |
| IIa-10 | (2) Cl, (4) CF₃ | 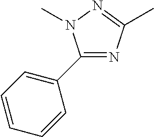 | OCH₃ |
| IIa-11 | (2) Cl | 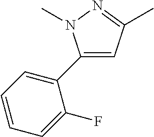 | OCH₃ |
| IIa-12 | — | 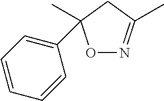 | OC₂H₅ |
| IIa-13 | (2) Cl, (4) Cl | 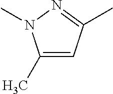 | OC₂H₅ |
| IIa-14 | (2) Cl, (4) Cl | 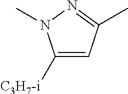 | OC₂H₅ |
| IIa-15 | (2) Cl, (4) Cl | 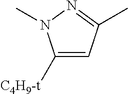 | OC₂H₅ |
| IIa-16 | (2) Cl, (4) Cl | 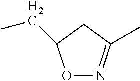 | OC₂H₅ |
| IIa-17 | (2) Cl, (4) Cl | 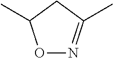 | OC₂H₅ |
| IIa-18 | — | 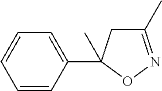 | OH |

Examples of the compounds of the formula (IIb) which are very particularly preferred as herbicide safeners according to the invention are listed in the table below.

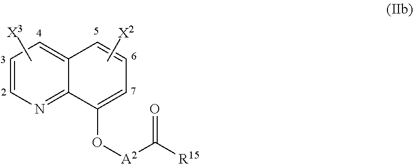

TABLE
Examples of compounds of the formula (IIb)

| Example | (Position) | (Position) | | |
|---|---|---|---|---|
| No. | X² | X³ | A² | R¹⁵ |
| IIb-1 | (5) | — | CH₂ | OH |
| | Cl | | | |
| IIb-2 | (5) | — | CH₂ | OCH₃ |
| | Cl | | | |
| IIb-3 | (5) | — | CH₂ | OC₂H₅ |
| | Cl | | | |
| IIb-4 | (5) | — | CH₂ | OC₃H₇-n |
| | Cl | | | |
| IIb-5 | (5) | — | CH₂ | OC₃H₇-i |
| | Cl | | | |
| IIb-6 | (5) | — | CH₂ | OC₄H₉-n |
| | Cl | | | |
| IIb-7 | (5) | — | CH₂ | OCH(CH₃)C₅H₁₁-n |
| | Cl | | | |
| IIb-8 | (5) | (2) | CH₂ | OH |
| | Cl | F | | |
| IIb-9 | (5) | (2) | CH₂ | OH |
| | Cl | Cl | | |
| IIb-10 | (5) | — | CH₂ | OCH₂CH═CH₂ |
| | Cl | | | |
| IIb-11 | (5) | — | CH₂ | OC₄H₉-i |
| | Cl | | | |
| IIb-12 | (5) Cl | — | CH₂ | 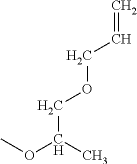 |
| IIb-13 | (5) Cl | — | 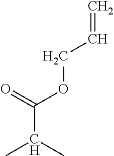 | OCH₂CH═CH₂ |
| IIb-14 | (5) Cl | — | 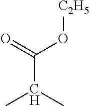 | OC₂H₅ |
| IIb-15 | (5) Cl | — | 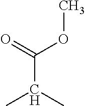 | OCH₃ |

Examples of the compounds of the formula (IIc) which are very particularly preferred as herbicide safeners according to the invention are listed in the table below.

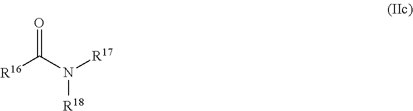

TABLE
Examples of compounds of the formula (IIc)

| Example | | |
|---|---|---|
| No. | R¹⁶ | N(R¹⁷,R¹⁸) |
| IIc-1 | CHCl₂ | N(CH₂CH═CH₂)₂ |
| IIc-2 | CHCl₂ | 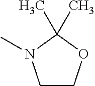 |
| IIc-3 | CHCl₂ | 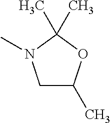 |
| IIc-4 | CHCl₂ | 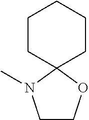 |
| IIc-5 | CHCl₂ | 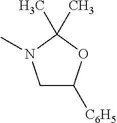 |
| IIc-6 | CHCl₂ | 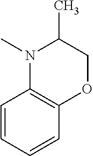 |
| IIc-7 | CHCl₂ | 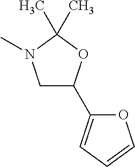 |

Examples of the compounds of the formula (ad) which are very particularly preferred as herbicide safeners according to the invention are listed in the table below.

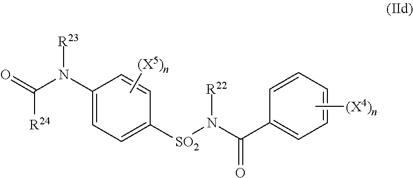

TABLE
Examples of compounds of the formula (IId)

| Example | | | | (Positions) | (Positions) |
|---|---|---|---|---|---|
| No. | R²² | R²³ | R²⁴ | (X⁴)ₙ | (X⁵)ₙ |
| IId-1 | H | H | CH₃ | (2) OCH₃ | — |
| IId-2 | H | H | C₂H₅ | (2) OCH₃ | — |
| IId-3 | H | H | C₃H₇-n | (2) OCH₃ | — |
| IId-4 | H | H | C₃H₇-i | (2) OCH₃ | — |
| IId-5 | H | H | 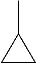 | (2) OCH₃ | — |
| IId-6 | H | H | CH₃ | (2) OCH₃ | — |
| | | | | (5) CH₃ | |
| IId-7 | H | H | C₂H₅ | (2) OCH₃ | — |
| | | | | (5) CH₃ | |
| IId-8 | H | H | C₃H₇-n | (2) OCH₃ | — |
| | | | | (5) CH₃ | |
| IId-9 | H | H | C₃H₇-i | (2) OCH₃ | — |
| | | | | (5) CH₃ | |
| IId-10 | H | H | 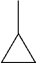 | (2) OCH₃ (5) CH₃ | — |
| IId-11 | H | H | OCH₃ | (2) OCH₃ | — |
| | | | | (5) CH₃ | |
| IId-12 | H | H | OC₂H₅ | (2) OCH₃ | — |
| | | | | (5) CH₃ | |
| IId-13 | H | H | OC₃H₇-i | (2) OCH₃ | — |
| | | | | (5) CH₃ | |
| IId-14 | H | H | SCH₃ | (2) OCH₃ | — |
| | | | | (5) CH₃ | |
| IId-15 | H | H | SC₂H₅ | (2) OCH₃ | — |
| | | | | (5) CH₃ | |
| IId-16 | H | H | SC₃H₇-i | (2) OCH₃ | — |
| | | | | (5) CH₃ | |
| IId-17 | H | H | NHCH₃ | (2) OCH₃ | — |
| | | | | (5) CH₃ | |
| IId-18 | H | H | NHC₂H₅ | (2) OCH₃ | — |
| | | | | (5) CH₃ | |
| IId-19 | H | H | NHC₃H₇-i | (2) OCH₃ | — |
| | | | | (5) CH₃ | |
| IId-20 | H | H | 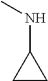 | (2) OCH₃ (5) CH₃ | — |
| IId-21 | H | H | NHCH₃ | (2) OCH₃ | — |
| IId-22 | H | H | NHC₃H₇-i | (2) OCH₃ | — |
| IId-23 | H | H | N(CH₃)₂ | (2) OCH₃ | — |
| IId-24 | H | H | N(CH₃)₂ | (3) CH₃ | — |
| | | | | (4) CH₃ | |
| IId-25 | H | H | CH₂—O—CH₃ | (2) OCH₃ | — |

Examples of the compounds of the formula (IIe) which are very particularly preferred as herbicide safeners according to the invention are listed in the table below.

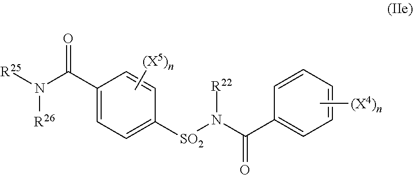

TABLE
Examples of compounds of the formula (IIe)

| Example | | | | (Positions) | (Positions) |
|---|---|---|---|---|---|
| No. | R²² | R²⁵ | R²⁶ | (X⁴)ₙ | (X⁵)ₙ |
| IIe-1 | H | H | CH₃ | (2) OCH₃ | — |
| IIe-2 | H | H | C₂H₅ | (2) OCH₃ | — |
| IIe-3 | H | H | C₃H₇-n | (2) OCH₃ | — |
| IIe-4 | H | H | C₃H₇-i | (2) OCH₃ | — |
| IIe-5 | H | H | 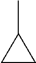 | (2) OCH₃ | — |
| IIe-6 | H | CH₃ | CH₃ | (2) OCH₃ | — |
| IIe-7 | H | H | CH₃ | (2) OCH₃ | — |
| | | | | (5) CH₃ | |
| IIe-8 | H | H | C₂H₅ | (2) OCH₃ | — |
| | | | | (5) CH₃ | |
| IIe-9 | H | H | C₃H₇-n | (2) OCH₃ | — |
| | | | | (5) CH₃ | |
| IIe-10 | H | H | C₃H₇-i | (2) OCH₃ | — |
| | | | | (5) CH₃ | |
| IIe-11 | H | H | 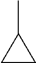 | (2) OCH₃ (5) CH₃ | — |
| IIe-12 | H | CH₃ | CH₃ | (2) OCH₃ | — |
| | | | | (5) CH₃ | |

Most preferred as crop plant compatibility-improving compound [component (b′)] are cloquintocet-mexyl, fenchlorazole-ethyl, isoxadifen-ethyl, mefenpyr-diethyl, furilazole, fenclorim, cumyluron, dymron, dimepiperate and the compounds IIe-5 and IIe-11, and particular emphasis is given to cloquintocet-mexyl and mefenpyr-diethyl.

The compounds of the general formula (IIa) to be used as safeners according to the invention are known and/or can be prepared by processes known per se (cf. WO-A-91/07874, WO-A-95/07897).

The compounds of the general formula (IIb) to be used as safeners according to the invention are known and/or can be prepared by processes known per se (cf. EP-A-191736).

The compounds of the general formula (IIc) to be used as safeners according to the invention are known and/or can be prepared by processes known per se (cf. DE-A-2218097, DE-A-2350547).

The compounds of the general formula (IId) to be used as safeners according to the invention are known and/or can be prepared by processes known per se (cf. DE-A-19621522/U.S. Pat. No. 6,235,680).

The compounds of the general formula (IIe) to be used as safeners according to the invention are known and can be prepared by processes known per se (cf. WO-A-99/66795/U.S. Pat. No. 6,251,827).

Examples of the selectively herbicidal combinations according to the invention comprising in each case one active compound of the formula (I) and in each case one of the safeners defined above are listed in the table below.

TABLE
Examples of combinations according to the invention

| Active compounds of the formula | |
|---|---|
| (I) | Safener |
| A.1 | cloquintocet-mexyl |
| A.1 | fenchlorazole-ethyl |
| A.1 | isoxadifen-ethyl |
| A.1 | mefenpyr-diethyl |
| A.1 | furilazole |
| A.1 | fenclorim |
| A.1 | cumyluron |
| A.1 | daimuron/dymron |
| A.1 | dimepiperate |
| A.1 | IIe-11 |
| A.1 | IIe-5 |
| A.2 | cloquintocet-mexyl |
| A.2 | fenchlorazole-ethyl |
| A.2 | isoxadifen-ethyl |
| A.2 | mefenpyr-diethyl |
| A.2 | furilazole |
| A.2 | fenclorim |
| A.2 | cumyluron |
| A.2 | daimuron/dymron |
| A.2 | dimepiperate |
| A.2 | IIe-11 |
| A.2 | IIe-5 |
| A.3 | cloquintocet-mexyl |
| A.3 | fenchlorazole-ethyl |
| A.3 | isoxadifen-ethyl |
| A.3 | mefenpyr-diethyl |
| A.3 | furilazole |
| A.3 | fenclorim |
| A.3 | cumyluron |
| A.3 | daimuron/dymron |
| A.3 | dimepiperate |
| A.3 | IIe-11 |
| A.3 | IIe-5 |
| A.4 | cloquintocet-mexyl |
| A.4 | fenchlorazole-ethyl |
| A.4 | isoxadifen-ethyl |
| A.4 | mefenpyr-diethyl |
| A.4 | furilazole |
| A.4 | fenclorim |
| A.4 | cumyluron |
| A.4 | daimuron/dymron |
| A.4 | dimepiperate |
| A.4 | IIe-11 |
| A.4 | IIe-5 |
| A.5 | cloquintocet-mexyl |
| A.5 | fenchlorazole-ethyl |
| A.5 | isoxadifen-ethyl |
| A.5 | mefenpyr-diethyl |
| A.5 | furilazole |
| A.5 | fenclorim |
| A.5 | cumyluron |
| A.5 | daimuron/dymron |
| A.5 | dimepiperate |
| A.5 | IIe-11 |
| A.5 | IIe-5 |
| A.6 | cloquintocet-mexyl |
| A.6 | fenchlorazole-ethyl |
| A.6 | isoxadifen-ethyl |
| A.6 | mefenpyr-diethyl |
| A.6 | furilazole |
| A.6 | fenclorim |
| A.6 | cumyluron |
| A.6 | daimuron/dymron |
| A.6 | dimepiperate |
| A.6 | IIe-11 |
| A.6 | IIe-5 |
| A.7 | cloquintocet-mexyl |
| A.7 | fenchlorazole-ethyl |
| A.7 | isoxadifen-ethyl |
| A.7 | mefenpyr-diethyl |
| A.7 | furilazole |
| A.7 | fenclorim |
| A.7 | cumyluron |
| A.7 | daimuron/dymron |
| A.7 | dimepiperate |
| A.7 | IIe-11 |
| A.7 | IIe-5 |
| A.8 | cloquintocet-mexyl |
| A.8 | fenchlorazole-ethyl |
| A.8 | isoxadifen-ethyl |
| A.8 | mefenpyr-diethyl |
| A.8 | furilazole |
| A.8 | fenclorim |
| A.8 | cumyluron |
| A.8 | daimuron/dymron |
| A.8 | dimepiperate |
| A.8 | IIe-11 |
| A.8 | IIe-5 |

Surprisingly, it has now been found that the active compound combinations defined above of 4′4′-dioxaspiro-spirocyclically substituted tetramates of the general formula (I) and safeners (antidotes) from the group (b′) set out above combine very good useful plant tolerance with a particularly high herbicidal activity and can be used in various crops, in particular in cereals (especially wheat), but also in soya, potatoes, maize and rice, for the selective control of weeds.

In this context it is to be considered surprising that, from a multiplicity of known safeners or antidotes capable of antagonizing the damaging effect of a herbicide on the crop plants, it is specifically the compounds of group (b′) set out above which are suitable for compensating—almost completely—the damaging effect of 4′4′-dioxaspiro-spirocyclically substituted tetramates on the crop plants, without at the same time having any critical adverse effect on the herbicidal activity against the weeds.

Emphasis may be given here to the particularly advantageous effect of the particularly preferred and most preferred combination partners from group (b′), in particular with regard to the gentle treatment of cereal plants, such as wheat, barley and rye, for example, but also maize and rice, as crop plants.

All the active compounds of the formula (I) present in the materials according to the invention can be prepared by processes described in the prior art (see references mentioned above). Their activity is good; however, in particular at low application rates and concentrations, it is not always satisfactory. Furthermore, the compatibility of these compounds with plants is not always sufficient. Accordingly, there is a need for a boost of action and/or an improved compatibility with plants with respect to crop plants of the crop protection compositions comprising the compounds.

In the literature it has already been described how the action of various active compounds can be boosted by addition of ammonium salts. The salts in question, however, are detersive salts (for example WO 95/017817) or salts which have relatively long alkyl substituents and/or aryl substituents and which have a permeabilizing action or which increase the active compound's solubility (for example EP-A 0 453 086, EP-A 0 664 081, FR-A 2 600 494, U.S. Pat. No. 4,844,734, U.S. Pat. No. 5,462,912, U.S. Pat. No. 5,538,937, US-A 03/0224939, US-A 05/0009880, US-A 05/0096386). Moreover, the prior art describes the action only for particular active compounds and/or particular applications of the corresponding compositions. A boost to action by ammonium sulphate, for example, is described by way of example for the herbicides glyphosate and phosphinothricin (U.S. Pat. No. 6,645,914, EP-A2 0 036 106).

The use of ammonium sulphate as a formulating assistant has also been described for certain active compounds and applications (WO 92/16108), but its purpose therein is to stabilize the formulation, not to boost the action.

Herbicidal and insecticidal compositions of cyclic ketoenols with ammonium salts or phosphonium salts for boosting the action are described in WO 07/068,427 and WO 07/068,428.

Surprisingly, it has now been found that the activity of herbicides and/or acaricides and/or insecticides from the class of the 4′4′-dioxaspiro-spirocyclically substituted tetramates can be increased significantly by adding ammonium salts or phosphonium salts to the application solution or by incorporating these salts into a formulation comprising 4′4′-dioxaspiro-spirocyclically substituted tetramates. The present invention therefore provides for the use of ammonium salts and/or phosphonium salts to boost the activity of crop protection compositions which comprise insecticidally and/or acaricidally and/or herbicidally active 4′4′-dioxaspiro-spirocyclically substituted tetramates as active compound. The invention also provides materials which comprise insecticidally and/or acaricidally and/or herbicidally active 4′4′-dioxaspiro-spirocyclically substituted tetramates and action-boosting ammonium or phosphonium salts, including specifically not only formulated active compounds but also ready-to-use materials (spray liquors). The invention additionally provides, finally, for the use of these materials for controlling insects and/or spider mites and/or unwanted plant growth.

The active compounds can be used in the compositions of the invention in a broad concentration range. The concentration of the active compounds in the formulation is typically 0.1%-50% by weight.

Ammonium salts and phosphonium salts which inventively boost the activity of crop protection compositions comprising 4′4′-dioxaspiro-spirocyclically substituted tetramates are defined by formula (II′)

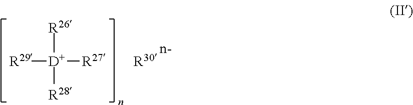

in which
D represents nitrogen or phosphorus,
D preferably represents nitrogen,
R^{26′}, R^{27′}, R^{28′} and R^{29′} independently of one another represent hydrogen or in each case optionally substituted C₁-C₈-alkyl or mono- or polyunsaturated, optionally substituted C₁-C₃-alkylene, the substituents being selectable from halogen, nitro and cyano,
R^{26′}, R^{27′}, R^{28′} and R^{29′} independently of one another preferably represent hydrogen or in each case optionally substituted C₁-C₄-alkyl, the substituents being selectable from halogen, nitro and cyano,
R^{26′}, R^{27′}, R^{28′} and R^{29′} independently of one another particularly preferably represent hydrogen, methyl, ethyl, n-propyl, isopropyl, n-butyl, isobutyl, sec-butyl or tert-butyl,
R^{26′}, R^{27′}, R^{28′} and R^{29′} very particularly preferably represent hydrogen,
R^{26′}, R^{27′}, R^{28′} and R^{29′} furthermore very particularly preferably simultaneously represent methyl or simultaneously represent ethyl,
n represents 1, 2, 3 or 4,
n preferably represents 1 or 2,
R^{30′} represents an organic or inorganic anion,
R^{30′} preferably represents hydrogencarbonate, tetraborate, fluoride, bromide, iodide, chloride, monohydrogenphosphate, dihydrogenphosphate, hydrogensulphate, tartrate, sulphate, nitrate, thiosulphate, thiocyanate, formate, lactate, acetate, propionate, butyrate, pentanoate or oxalate,
R^{30′} furthermore preferably represents carbonate, pentaborate, sulphite, benzoate, hydrogenoxalate, hydrogencitrate, methylsulphate or tetrafluoroborate,
R^{30′} particularly preferably represents lactate, sulphate, nitrate, thiosulphate, thiocyanate, citrate, oxalate or formate,
R^{30′} moreover particularly preferably represents acetate, monohydrogenphosphate or dihydrogenphosphate and
R^{30′} very particularly preferably represents sulphate, thiocyanate, dihydrogenphosphate or monohydrogenphosphate.

The ammonium salts and phosphonium salts of the formula (II') can be used in a broad concentration range to boost the activity of crop protection compositions comprising compounds of the formula (I). In general the ammonium salts or phosphonium salts are used in the ready-to-use crop protection composition in a concentration of 0.5 to 80 mmol/l, preferably 0.75 to 37.5 mmol/l, more preferably 1.5 to 25 mmol/l. In the case of a formulated product the ammonium salt and/or phosphonium salt concentration in the formulation is chosen such that it is within these stated general, preferred or particularly preferred ranges after the formulation has been diluted to the desired active-compound concentration. The concentration of the salt in the formulation is typically 1%-50% by weight.

In one preferred embodiment of the invention the activity is boosted by adding to the crop protection compositions not only an ammonium salt and/or phosphonium salt but also, additionally, a penetrant. It is considered entirely surprising that even in these cases an even greater boost to activity is observed. The present invention therefore also provides for the use of a combination of penetrant and ammonium salts and/or phosphonium salts to boost the activity of crop protection compositions which comprise herbicidally active 4′4′-dioxaspiro-spirocyclically substituted tetramates as active compound. The invention also provides materials which comprise insecticidally and/or acaricidally and/or herbicidally active 4′4′-dioxaspiro-spirocyclically substituted tetramates, penetrants and ammonium salts and/or phosphonium salts, including specifically not only formulated active compounds but also ready-to-use materials (spray liquors). The invention additionally provides, finally, for the use of these materials for controlling insects and/or spider mites and/or unwanted plant growth.

Suitable penetrants in the present context include all those substances which are typically used to enhance the penetration of active agrochemical compounds into plants. Penetrants are defined in this context by their ability to penetrate from the aqueous spray liquor and/or from the spray coating into the cuticle of the plant and thereby to increase the mobility of active compounds in the cuticle. The method described in the literature (Baur et al., 1997, Pesticide Science 51, 131-152) can be used in order to determine this property.

Suitable penetrants are, for example, alkanol alkoxylates. Penetrants according to the invention are alkanol alkoxylates of the formula

R—O—(-AO)ᵥ—R′ (III′)

in which
R represents straight-chain or branched alkyl having 4 to 20 carbon atoms,
R′ represents hydrogen, methyl, ethyl, n-propyl, isopropyl, n-butyl, isobutyl, t-butyl, n-pentyl or n-hexyl,
AO represents an ethylene oxide radical, a propylene oxide radical, a butylene oxide radical or represents mixtures of ethylene oxide and propylene oxide radicals or butylene oxide radicals and
v represents numbers from 2 to 30.

Here, alkanol alkoxylates in which R′ represents hydrogen are referred to as “open” alkanol alkoxylates. A preferred group of penetrants are alkanol alkoxylates of the formula

R—O—(-EO—)ₙ—R′ (III′-a)

in which
R has the meaning given above,
R′ has the meaning given above,
EO represents —CH₂—CH₂—O— and
n represents numbers from 2 to 20.

A further preferred group of penetrants are alkanol alkoxylates of the formula

R—O—(-EO—)ₚ—(—PO—)_{q}—R′ (III′-b)

in which
R has the meaning given above,
R′ has the meaning given above,
EO represents —CH₂—CH₂—O—,
PO represents

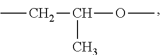

represents numbers from 1 to 10 and
q represents numbers from 1 to 10.

A further preferred group of penetrants are alkanol alkoxylates of the formula

R—O—(—PO—)ᵣ-(EO—)ₛ—R′ (III′-c)

in which
R has the meaning given above,
R′ has the meaning given above,
EO represents —CH₂—CH₂—O—,
PO represents

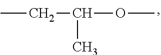

r represents numbers from 1 to 10 and
s represents numbers from 1 to 10.

A further preferred group of penetrants are alkanol alkoxylates of the formula

R—O—(-EO—)ₚ—(—BO—)_{q}—R′ (III′-d)

in which
R and R′ have the meanings given above,
EO represents CH₂—CH₂—O—,
BO represents

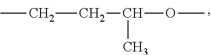

p represents numbers from 1 to 10 and
q represents numbers from 1 to 10.

A further preferred group of penetrants are alkanol alkoxylates of the formula

R—O—(—BO—)ᵣ—(-EO—)ₛ—R′ (III′-e)

in which
R and R′ have the meanings given above,
BO represents

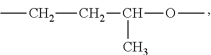

EO represents CH₂—CH₂—O—,
r represents numbers from 1 to 10 and
s represents numbers from 1 to 10.

A further preferred group of penetrants are alkanol alkoxylates of the formula

CH₃—(CH₂)ₜ—CH₂—O—(—CH₂—CH₂—O—)ᵤ—R′ (III′-f)

in which
R′ has the meaning given above,
t represents numbers from 8 to 13
u represents numbers from 6 to 17.

In the formulae indicated above,
R preferably represents butyl, isobutyl, n-pentyl, isopentyl, neopentyl, n-hexyl, isohexyl, n-octyl, isooctyl, 2-ethylhexyl, nonyl, isononyl, decyl, n-dodecyl, isododecyl, lauryl, myristyl, isotridecyl, trimethylnonyl, palmityl, stearyl or eicosyl.

As an example of an alkanol alkoxylate of the formula (III-c) mention may be made of 2-ethylhexyl alkoxylate of the formula

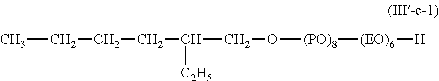

in which
EO represents —CH₂—CH₂—O—,
PO represents

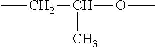

and
the numbers 8 and 6 represent average values.

As an example of an alkanol alkoxylate of the formula (III-d) mention may be made of the formula

CH₃—(CH₂)₁₀—O—(-EO—)₆—(—BO—)₂—CH₃ (III′-d-1)

in which
EO represents CH₂—CH₂—O—,
BO represents

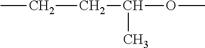

and
the numbers 10, 6 and 2 represent average values.

Particularly preferred alkanol alkoxylates of the formula (III′-f) are compounds of this formula in which
t represents numbers from 9 to 12 and
u represents numbers from 7 to 9.

Mention may be made with very particular preference of alkanol alkoxylate of the formula (III′-f-1)

CH₃—(CH₂)ₜ—CH₂—O—(—CH₂—CH₂—O—)ᵤ—H (III′-f-1)

in which
t represents the average value 10.5 and
u represents the average value 8.4.

A general definition of the alkanol alkoxylates is given by the formulae above. These substances are mixtures of compounds of the stated type with different chain lengths. The indices therefore have average values which may also deviate from whole numbers.

The alkanol alkoxylates of the formulae stated are known and in some cases are available commercially or can be prepared by known methods (cf. WO 98/35 553, WO 00/35 278 and EP-A 0 681 865).

Suitable penetrants also include, for example, substances which promote the availability of the compounds of the formula (I) in the spray coating. These include, for example, vegetable oils, mineral oils, paraffin oils and fatty acid esters.

Vegetable oils are generally known and commercially available. The term vegetable oils is to be understood as including, for example, oils from oleaginous plant species, such as soya bean oil, rapeseed oil, maize germ oil, maize kernel oil, sunflower oil, cottonseed oil, linseed oil, coconut oil, palm oil, thistle oil, walnut oil, arachis oil, olive oil, castor oil or colza oil, in particular soya bean oil, rapeseed oil, maize germ oil or sunflower oil and mixtures thereof. The vegetable oils (triglycerides) are preferably esters of C₁₀-C₂₂—, preferably C₁₂-C₂₀—, fatty acids of glycerol. The C₁₀-C₂₂-fatty acid esters of glycerol are, for example, esters of unsaturated or saturated C₁₂-C₂₀— fatty acids, in particular those having an even number of carbon atoms, for example erucic acid, lauric acid, palmitic acid, and in particular C₁₈-fatty acids, such as stearic acid, oleic acid, linoleic acid or linolenic acid.

Suitable mineral oils are various commercially available distillate fractions of mineral oil (petroleum). Preference is given to mixtures of open-chain C₁₄-C₃₀-hydrocarbons, cyclic hydrocarbons (naphthenes) and aromatic hydrocarbons. The hydrocarbons can be either straight-chain or branched. Particular preference is given to mixtures having an aromatic portion of less than 8% by weight. Very particular preference is given to mixtures having an aromatic portion of less than 4% by weight.

Suitable paraffin oils are straight-chain and branched C₁₄-C₃₀-hydrocarbons. Paraffin oils are also known as base oil or white oil and are commercially available, for example, as Bayol® 85 (Exxon Mobil, Machelen, Belgium), Marcol® 82 (Exxon Mobil, Machelen, Belgium), BAR 0020 (RA.M.oil S.p.A., Naples, Italy), Pionier 0032-20 (Hansen & Rosenthal KG, Hamburg, Germany) or, for example, Kristol M14 (Carless, Surrey, England).

Suitable fatty acid esters are alkyl fatty acid esters, such as C₁-C₂₀-alkyl C₁₀-C₂₂-fatty acid esters. Preference is given to methyl esters, ethyl esters, propyl esters, butyl esters, 2-ethylhexyl esters and dodecyl esters. Particular preference is given to methyl esters and ethyl esters. Examples of synthetic fatty acid esters are, for example, those which are derived from fatty acids having an odd number of carbon atoms, such as C₁₁-C₂₁-fatty acid esters. The transesterification can be carried out by known methods, as described, for example, in Römpp Chemie Lexikon, 9th Edition, Volume 2, page 1343, Thieme Verlag, Stuttgart. In the adjuvant compositions according to the invention, the fatty acid esters can be present in the form of commercially available esters, in particular esters such as rapeseed oil methyl ester, for example Edenor® MESU (Cognis, Germany) or the Agnique® ME series (Cognis, Germany) or in the form of commercially available oil-containing formulation additives, in particular those based on rapeseed oil methyl ester or rapeseed oil ethyl ester, for example Hasten® (Victoria Chemicals, Australia), Actirob® B (Novance, France) or Stefes Mero® (Stefes, Germany).

The concentration of penetrant in the materials according to the invention can be varied within a wide range. In the case of a formulated crop protection composition it is in general 1% to 95%, preferably 1% to 55%, more preferably 15%-40% by weight. In the ready-to-use materials (spray liquors) the concentrations are generally between 0.1 and 10 g/l, preferably between 0.5 and 5 g/l.

Inventively emphasized combinations of active compound, salt and penetrant are listed in the table below. “As per test” means here that any compound that acts as a penetrant in the cuticle penetration test (Baur et al., 1997, Pesticide Science 51, 131-152) is suitable.

| | Active | | |
|---|---|---|---|
| # | compound | Salt | Penetrant |
| 1 | A.1 | ammonium sulphate | as per test |
| 2 | A.1 | ammonium lactate | as per test |
| 3 | A.1 | ammonium nitrate | as per test |
| 4 | A.1 | ammonium thiosulphate | as per test |
| 5 | A.1 | ammonium thiocyanate | as per test |
| 6 | A.1 | ammonium citrate | as per test |
| 7 | A.1 | ammonium oxalate | as per test |
| 8 | A.1 | ammonium formate | as per test |
| 9 | A.1 | ammonium hydrogenphosphate | as per test |
| 10 | A.1 | ammonium dihydrogenphosphate | as per test |
| 11 | A.1 | ammonium carbonate | as per test |
| 12 | A.1 | ammonium benzoate | as per test |
| 13 | A.1 | ammonium sulphite | as per test |
| 14 | A.1 | ammonium benzoate | as per test |
| 15 | A.1 | ammonium hydrogenoxalate | as per test |
| 16 | A.2 | ammonium sulphate | as per test |
| 17 | A.2 | ammonium lactate | as per test |
| 18 | A.2 | ammonium nitrate | as per test |
| 19 | A.2 | ammonium thiosulphate | as per test |
| 20 | A.2 | ammonium thiocyanate | as per test |
| 21 | A.2 | ammonium citrate | as per test |
| 22 | A.2 | ammonium oxalate | as per test |
| 23 | A.2 | ammonium formate | as per test |
| 24 | A.2 | ammonium hydrogenphosphate | as per test |
| 25 | A.2 | ammonium dihydrogenphosphate | as per test |
| 26 | A.2 | ammonium carbonate | as per test |
| 27 | A.2 | ammonium benzoate | as per test |
| 28 | A.2 | ammonium sulphite | as per test |
| 29 | A.2 | ammonium benzoate | as per test |
| 30 | A.2 | ammonium hydrogenoxalate | as per test |
| 31 | A.3 | ammonium sulphate | as per test |
| 32 | A.3 | ammonium lactate | as per test |
| 33 | A.3 | ammonium nitrate | as per test |
| 34 | A.3 | ammonium thiosulphate | as per test |
| 35 | A.3 | ammonium thiocyanate | as per test |
| 36 | A.3 | ammonium citrate | as per test |
| 37 | A.3 | ammonium oxalate | as per test |
| 38 | A.3 | ammonium formate | as per test |
| 39 | A.3 | ammonium hydrogenphosphate | as per test |
| 40 | A.3 | ammonium dihydrogenphosphate | as per test |
| 41 | A.3 | ammonium carbonate | as per test |
| 42 | A.3 | ammonium benzoate | as per test |
| 43 | A.3 | ammonium sulphite | as per test |
| 44 | A.3 | ammonium benzoate | as per test |
| 45 | A.3 | ammonium hydrogenoxalate | as per test |
| 46 | A.4 | ammonium sulphate | as per test |
| 47 | A.4 | ammonium lactate | as per test |
| 48 | A.4 | ammonium nitrate | as per test |
| 49 | A.4 | ammonium thiosulphate | as per test |
| 50 | A.4 | ammonium thiocyanate | as per test |
| 51 | A.4 | ammonium citrate | as per test |
| 52 | A.4 | ammonium oxalate | as per test |
| 53 | A.4 | ammonium formate | as per test |
| 54 | A.4 | ammonium hydrogenphosphate | as per test |
| 55 | A.4 | ammonium dihydrogenphosphate | as per test |
| 56 | A.4 | ammonium carbonate | as per test |
| 57 | A.4 | ammonium benzoate | as per test |
| 58 | A.4 | ammonium sulphite | as per test |
| 59 | A.4 | ammonium benzoate | as per test |
| 60 | A.4 | ammonium hydrogenoxalate | as per test |
| 61 | A.5 | ammonium sulphate | as per test |
| 62 | A.5 | ammonium lactate | as per test |
| 63 | A.5 | ammonium nitrate | as per test |
| 64 | A.5 | ammonium thiosulphate | as per test |
| 65 | A.5 | ammonium thiocyanate | as per test |
| 66 | A.5 | ammonium citrate | as per test |
| 67 | A.5 | ammonium oxalate | as per test |
| 68 | A.5 | ammonium formate | as per test |
| 69 | A.5 | ammonium hydrogenphosphate | as per test |
| 70 | A.5 | ammonium dihydrogenphosphate | as per test |
| 71 | A.5 | ammonium carbonate | as per test |
| 72 | A.5 | ammonium benzoate | as per test |
| 73 | A.5 | ammonium sulphite | as per test |
| 74 | A.5 | ammonium benzoate | as per test |
| 75 | A.5 | ammonium hydrogenoxalate | as per test |
| 76 | A.6 | ammonium sulphate | as per test |
| 77 | A.6 | ammonium lactate | as per test |
| 78 | A.6 | ammonium nitrate | as per test |
| 79 | A.6 | ammonium thiosulphate | as per test |
| 80 | A.6 | ammonium thiocyanate | as per test |
| 81 | A.6 | ammonium citrate | as per test |
| 82 | A.6 | ammonium oxalate | as per test |
| 83 | A.6 | ammonium formate | as per test |
| 84 | A.6 | ammonium hydrogenphosphate | as per test |
| 85 | A.6 | ammonium dihydrogenphosphate | as per test |
| 86 | A.6 | ammonium carbonate | as per test |
| 87 | A.6 | ammonium benzoate | as per test |
| 88 | A.6 | ammonium sulphite | as per test |
| 89 | A.6 | ammonium benzoate | as per test |
| 90 | A.6 | ammonium hydrogenoxalate | as per test |
| 91 | A.7 | ammonium sulphate | as per test |
| 92 | A.7 | ammonium lactate | as per test |
| 93 | A.7 | ammonium nitrate | as per test |
| 94 | A.7 | ammonium thiosulphate | as per test |
| 95 | A.7 | ammonium thiocyanate | as per test |
| 96 | A.7 | ammonium citrate | as per test |
| 97 | A.7 | ammonium oxalate | as per test |
| 98 | A.7 | ammonium formate | as per test |
| 99 | A.7 | ammonium hydrogenphosphate | as per test |
| 100 | A.7 | ammonium dihydrogenphosphate | as per test |
| 101 | A.7 | ammonium carbonate | as per test |
| 102 | A.7 | ammonium benzoate | as per test |
| 103 | A.7 | ammonium sulphite | as per test |
| 104 | A.7 | ammonium benzoate | as per test |
| 105 | A.7 | ammonium hydrogenoxalate | as per test |
| 106 | A.8 | ammonium sulphate | as per test |
| 107 | A.8 | ammonium lactate | as per test |
| 108 | A.8 | ammonium nitrate | as per test |
| 109 | A.8 | ammonium thiosulphate | as per test |
| 110 | A.8 | ammonium thiocyanate | as per test |
| 111 | A.8 | ammonium citrate | as per test |
| 112 | A.8 | ammonium oxalate | as per test |
| 113 | A.8 | ammonium formate | as per test |
| 114 | A.8 | ammonium hydrogenphosphate | as per test |
| 115 | A.8 | ammonium dihydrogenphosphate | as per test |
| 116 | A.8 | ammonium carbonate | as per test |
| 117 | A.8 | ammonium benzoate | as per test |
| 118 | A.8 | ammonium sulphite | as per test |
| 119 | A.8 | ammonium benzoate | as per test |
| 120 | A.8 | ammonium hydrogenoxalate | as per test |

Suitable for use as solvents in the compositions according to the invention are all water-miscible solvents which are customarily mentioned for agrochemical formulations and in which the active compounds of the formulae (I) and (I′) are soluble at the concentrations used herein. Examples which may be mentioned are water, alcohols, such as methanol, ethanol or isopropanol, ethers or polyethers, such as 1,4-dioxane, tetrahydrofuran or dimethoxyethane, amides, such as formamide, acetamide, N,N-dimethylformamide, N,N-dimethylacetamide or Hallcomid® (mixture of 50-60% N,N-dimethyloctanamide and 35-45% N,N-dimethyldecanamide), sulphoxides/sulphones, such as dimethyl sulphoxide or sulpholane, and lactones/lactams, such as N-methylpyrrolidone and gamma-butyrolactone.

The preferred solvent is water.

To prepare the water-soluble concentrates according to the invention comprising a compound of the formula (I), it is advantageous to generate the compound of the formula (I) in situ during the preparation of the composition by reacting the corresponding compound of the formula (I′) with a suitable base.

This process allows the preparation of compositions comprising compounds of the formula (I) whose stability in isolated form is sometimes lower. Suitable bases are, in principle, all organic and inorganic bases, provided their use for agricultural purposes is acceptable.

Examples of bases are
a) metal hydroxides, such as, for example, lithium hydroxide, sodium hydroxide and potassium hydroxide, magnesium hydroxide and calcium hydroxide, aluminium hydroxide, zinc hydroxide or copper hydroxide,
b) metal oxides, such as, for example, lithium oxide, sodium oxide and potassium oxide or aluminium oxide,
c) amines of the general formula NR¹R²R³. Here, R¹, R² and R³ may be identical or different and in each case represent hydrogen, C₁-C₅-alkyl, C₁-C₅-isoalkyl or C₃-C₇-cycloalkyl which for their part may in each case be mono- or polysubstituted by fluorine, chlorine, bromine, cyano, hydroxyl or interrupted by one or more oxygen or sulphur atoms.
   Specific examples are ammonia, methylamine, dimethylamine, triethylamine, ethylamine, diethylamine, triethylamine, isopropylamine, ethanolamine, diethanolamine, triethanolamine, 2-diethylaminoethanol, diisopropylamine, cyclohexylamine, dicyclohexylamine,
d) mono-, bi- or tricyclic amines, such as, for example, morpholine, thiomorpholine, piperidine, pyrrolidine, 1,4-diazabicyclo[2.2.2]octane (DABCO) or 1,5-diazabicyclo[4.3.0]undec-7-ene (DBU),
e) diamines, such as, for example, N,N-bis(2-hydroxyethyl)-C₈-C₁₈-alkylamines, hexa-methylenetetramine, N,N,N′,N′-tetrakis(2-hydroxypropyl)ethylenediamine, 2-diethylaminoethylamine, N,N,N′,N′-tetraethylethylenediamine, N,N,N′,N′-tetramethylethylenediamine, 2-(2-aminoethylamino)ethanol or lysine,
f) aromatic amines, such as, for example, pyridine, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, pyrrole, imidazole, quinoline, quinoxaline, 1,2-dimethylimidazole, 1,3-dimethylimidazolium methylsulphate,
g) carbonates, such as, for example, potassium carbonate, potassium bicarbonate, sodium carbonate, sodium bicarbonate, magnesium carbonate, calcium carbonate, copper carbonate, zinc carbonate or lithium carbonate,
h) sulphites, such as, for example, sodium sulphite, potassium sulphite, lithium sulphite or zinc sulphite,
i) phosphates, such as, for example, lithium phosphate, potassium phosphate, sodium phosphate, calcium phosphate and magnesium phosphate, lithium hydrogenphosphate, potassium hydrogenphosphate, sodium hydrogenphosphate, calcium hydrogenphosphate and magnesium hydrogenphosphate or potassium dihydrogenphosphate and sodium dihydrogenphosphate,
j) alkoxides, such as, for example, lithium methoxide, sodium methoxide and potassium methoxide or lithium ethoxide, sodium ethoxide and potassium ethoxide,
k) ammonium hydroxides, such as, for example, trimethylammonium hydroxide, triethylammonium hydroxide, tripropylammonium hydroxide or tributylammonium hydroxide, tetramethylammonium hydroxide, tetraethylammonium hydroxide, tetraethanolammonium hydroxide or methyltriethylammonium hydroxide,
l) amidines and guanidines which may in each case be substituted, for example acetamidine, formamidine, guanidine, 1,1,3,3-tetraminoguanides, aminoguanidine or arginine,
m) basic carboxylic acid salts, preferably acetates, such as, for example, lithium acetate, sodium acetate or potassium acetate, oxalates, such as, for example, sodium oxalate or potassium oxalate, tartrates, such as sodium tartrate or potassium tartrate, and also citrates, such as, for example, sodium citrate or potassium citrate,
n) strongly or weakly basic anion exchangers charged with hydroxide ions, for example those which are commercially available under the names AMBERLITE®, AMBERLYST®, DUOLITE®, DOWEX® or LEWATITE®.
o) basic ammonium salts (such as, for example, diammonium hydrogenphosphate).

In principle, the base may also be used in immobilized form, where the carrier material may be removed, for example by filtration, after the preparation of the concentrate according to the invention.

If required—based on the amount of the compounds of the general formula (I′) used—between 0.1 and 100 molar equivalents, typically from 0.5 to 3 molar equivalents, of the base may be employed.

Preferred as basic auxiliaries are lithium hydroxide, sodium hydroxide, potassium hydroxide, magnesium hydroxide, calcium hydroxide and also ammonia, basic ammonium salts (such as for example diammonium hydrogen phosphate) alkylamines and hydroxyalkylamines.

The formulations according to the invention optionally comprise further additives from the group of the antifoams, the preservatives, the spreading agents, the antifreeze agents, the antioxidants and/or the colorants.

Suitable preservatives are all substances which are usually employed for this purpose in agrochemical materials of this type. Examples which may be mentioned are Preventol® (Bayer AG) and Proxel®.

Suitable spreading agents are all substances which are usually employed for this purpose in agrochemical materials. Preference is given to polyether- or organo-modified polysiloxanes.

Suitable antifreeze agents are all substances of this type which are usually employed in agrochemical materials. Preference is given to urea, glycerol or propylene glycol.

Suitable antifoams are all substances which are usually employed for this purpose in agrochemical materials. Preference is given to polydimethylsiloxanes, silicone oils and magnesium stearate.

Suitable antioxidants are all substances which are usually employed for this purpose in agrochemical materials. Preference is given to butylated hydroxytoluene (2,6-di-t-butyl-4-methylphenol, BHT).

Suitable colorants are all substances which are usually employed for this purpose in agrochemical materials. Examples which may be mentioned are titanium dioxide, carbon black, zinc oxide and blue pigments, and also Permanent Red FOR.

In the formulations according to the invention the content of active compound is generally from 0.1 to 50% by weight, preferably from 1 to 25% by weight, particularly preferably from 2 to 20% by weight.

In the formulations according to the invention, the content of surfactant (active compound, if appropriate corrected for water content) is generally from 5 to 50% by weight and preferably from 10 to 30% by weight.

In the ready-to-use formulations (spray liquors), the content of surfactant is generally from 0.1 to 10 g/l, preferably from 0.3 to 3 g/l.

The surfactant is generally applied at an application rate of from 20 to 1000 g of a.i./ha, preferably from 100 to 300 g of a.i/ha.

Using, for example, according to process (A) ethyl N-(2,6-dimethyl-4-chloro-4,4-ethylenedioxy-phenylacetyl)-1-aminocyclohexanecarboxylate as starting material, the course of the process according to the invention can be represented by the reaction scheme below:

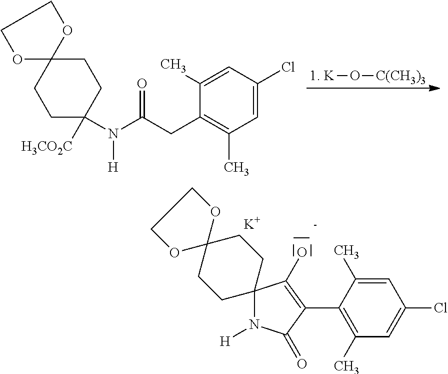

Using, for example, according to process (B) 8,8′-ethylenedioxy-3-[(2,6-dimethyl-4-chloro)-phenyl]-1-azaspiro[4,5]-decane-2,4-dione and NaOH as components, the course of the process according to the invention can be represented by the reaction scheme below:

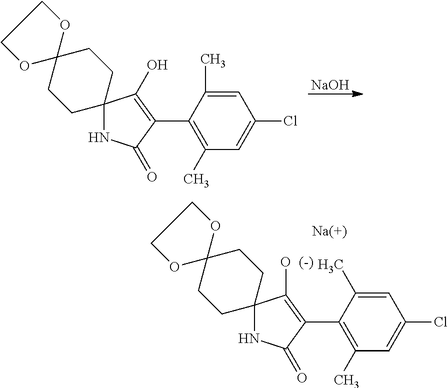

Some of the compounds, required as starting materials in the process (A) according to the invention, of the formula (II)

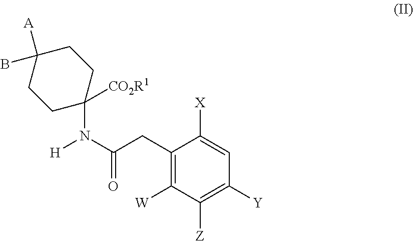

in which

A, B, W, X, Y, Z and R¹ have the meanings given above,

are known from WO 06/089633, or they can be prepared by the processes described therein.

The metal hydroxides, metal alkoxides or metal hydrides of the formulae (III) and (IV) furthermore required as starting materials for carrying out the process (B-α) according to the invention are generally known compounds of inorganic chemistry.

The amines of the formula (V) or ammonium compounds of the formula (VI) furthermore required as starting materials for carrying out the process (B-β) according to the invention are generally known compounds of organic chemistry.

In addition, the compounds of the formulae (I′) and (II) are known from the patent applications cited at the outset, and/or they can be prepared by the methods given therein.

The process (A) is characterized in that compounds of the formula (II) in which A, B, W, X, Y, Z and R¹ have the meanings given above are subjected to an intramolecular condensation in the presence of a base.

Suitable diluents for use in the process (A) according to the invention are all inert organic solvents. Preference is given to using hydrocarbons, such as toluene and xylene, furthermore ethers, such as dibutyl ether, tetrahydrofuran, dioxane, glycol dimethyl ether and diglycol dimethyl ether, moreover polar solvents, such as dimethyl sulphoxide, sulpholane, dimethylformamide and N-methylpyrrolidone, and also alcohols, such as methanol, ethanol, propanol, isopropanol, butanol, isobutanol and tert-butanol.

Suitable bases (deprotonating agents) for carrying out the process (A) according to the invention are all customary proton acceptors. Preference is given to using alkali metal and alkaline earth metal oxides, hydroxides and carbonates, such as sodium hydroxide, potassium hydroxide, magnesium oxide, calcium oxide, sodium carbonate, potassium carbonate and calcium carbonate, which can also be used in the presence of phase-transfer catalysts, such as, for example, triethylbenzylammonium chloride, tetrabutylammonium bromide, Adogen 464 (=methyl-trialkyl(C₈-C₁₀)ammonium chloride) or TDA 1 (=tris(methoxyethoxyethyl)amine). It is furthermore possible to use alkali metals, such as sodium or potassium. It is also possible to employ alkali metal and alkaline earth metal amides and hydrides, such as sodium amide, sodium hydride and calcium hydride, and additionally also alkali metal alkoxides, such as sodium methoxide, sodium ethoxide and potassium tert-butoxide.

When carrying out the process (A) according to the invention, the reaction temperatures can be varied within a relatively wide range. In general, the process is carried out at temperatures between 0° C. and 250° C., preferably between 50° C. and 150° C.

The process (A) according to the invention is generally carried out under atmospheric pressure.

When carrying out the process (A) according to the invention, the reaction components of the formula (II) and the deprotonating bases are generally employed in approximately doubly equimolar amounts. However, it is also possible to use a relatively large excess (up to 3 mol) of one component or the other.

The process (B) is characterized in that compounds of the formula (I′) are reacted with metal hydroxides or metal alkoxides of the formula (III) or metal hydrides of the formula (IV), if appropriate in the presence of a diluent.

Preferred diluents for use in the process (B) according to the invention are ethers, such as tetrahydrofuran, dioxane, diethyl ether or else alcohols, such as methanol, ethanol, isopropanol; however, it is also possible to use water.

The process (B) according to the invention is generally carried out under atmospheric pressure.

The reaction temperatures are generally between −20° C. and 100° C., preferably between 0° C. and 50° C.

The active compounds/active compound combinations according to the invention are well tolerated by plants, have favourable toxicity to warm-blooded species, show good environmental compatibility and are suitable for protecting plants and plant organs, for increasing yields, for improving the quality of the harvested crop and for controlling animal pests, in particular insects, arachnids, helminths, nematodes and molluscs, which are found in agriculture, in horticulture, in animal breeding, in forests, in gardens and leisure facilities, in the protection of stored products and materials, and in the hygiene sector. They can preferably be employed as plant protection agents. They are active against normally sensitive and resistant species and against all or individual developmental stages. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa.

From the class of the Gastropoda, for example, Anion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

Protozoa, such as Eimeria, can also be controlled.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example Scutigerella immaculata.

From the order of the Thysanoptera, for example, Baliothrips biformis, Enneothrips flavens, Frankliniella spp., Heliothrips spp., Hercinothrips femoralis, Rhipiphorothrips cruentatus, Scirtothrips spp., Taeniothrips cardamoni, Thrips spp.

From the order of the Thysanura, for example, Lepisma saccharina.

The plant-parasitic nematodes include, for example, Aphelenchoides spp., Bursaphelenchus spp., Ditylenchus dipsaci, Globodera spp., Heterodera spp., Longidorus spp., Meloidogyne spp., Pratylenchus spp., Radopholus similis, Trichodorus spp., Tylenchulus semipenetrans, Xiphinema spp.

In certain concentrations, or at certain application rates, the compounds/active compound combinations according to the invention can, if appropriate, also be used as herbicides, safeners, growth regulators or agents for improving the plant characteristics, or as microbicides, for example as fungicides, antimycotics, bactericides, viricides (including as agents against viroids) or as agents against MLOs (mycoplasma-like organisms) and RLOs (rickettsia-like organisms). If appropriate, they can also be used as intermediates or precursors for the synthesis of other active compounds.

According to the invention, it is possible to treat all plants and parts of plants. Plants are to be understood here as meaning all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment according to the invention of the plants and parts of plants with the active compounds/active compound combinations is carried out directly or by action on their environment, habitat or storage area according to customary treatment methods, for example by dipping, spraying, evaporating, atomizing, broadcasting, brushing-on, injecting and, in the case of propagation material, in particular in the case of seeds, furthermore by one- or multi-layer coating.

The active compounds/active compound combinations can be converted into the customary formulations such as solutions, emulsions, wettable powders, water- and oil-based suspensions, powders, dusts, pastes, soluble powders, soluble granules, granules for broadcasting, suspension-emulsion concentrates, natural and synthetic materials impregnated with active compound, fertilizers and also microencapsulations in polymeric materials.

These formulations are produced in a known manner, for example by mixing the active compounds/active compound combinations with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are:

for example ammonium salts and ground natural minerals such as kaolins, clays, talc, chalk, quartz, attapulgite, montmorillonite or diatomaceous earth, and ground synthetic minerals such as highly disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignosulphite waste liquors and methylcellulose.

Tackifiers such as carboxymethylcellulose, natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compound/active compound combination according to the invention can be present in its commercially available formulations and in the use forms, prepared from these formulations, as a mixture with other active compounds, such as insecticides, attractants, sterilants, bactericides, acaricides, nematicides, fungicides, growth-regulating substances, herbicides, safeners, fertilizers or semiochemicals.

A mixture with other known active compounds such as herbicides, fertilizers, growth-regulating substances, safeners, semiochemicals, or else with agents to improve the properties of the plant, is also possible.

When used as insecticides, the active compounds/active compound combinations according to the invention can furthermore be present in their commercially available formulations and in the use forms, prepared from these formulations, as a mixture with synergists. Synergists are compounds which increase the action of the active compounds, without it being necessary for the synergist added to be active itself.

When used as insecticides, the active compounds/active compound combinations according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

Application is carried out in a customary manner appropriate for the use forms.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

The method of treatment according to the invention is preferably used on genetically modified organisms, for example plants or plant parts.

Genetically modified plants (or transgenic plants) are plants in which a heterologous gene has been stably integrated into the genome.

The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology or RNA interference—RNAi—technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, the following effects, which exceed the effects which were actually to be expected, are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

At certain application rates, the active compounds/active compound combinations according to the invention may also have a strengthening effect in plants. Accordingly, they are also suitable for mobilizing the defence system of the plant against attack by unwanted phytopathogenic fungi and/or microorganisms and/or viruses. This may, if appropriate, be one of the reasons for the enhanced activity of the combinations according to the invention, for example against fungi. Plant-strengthening (resistance-inducing) substances are to be understood as meaning, in the present context, those substances or combinations of substances which are capable of stimulating the defence system of plants in such a way that, when subsequently inoculated with unwanted phytopathogenic fungi and/or microorganisms and/or viruses, the treated plants display a substantial degree of resistance to these unwanted phytopathogenic fungi and/or microorganisms and/or viruses. In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants which are also preferably treated according to the invention are resistant against one or more biotic stresses, i.e. said plants have a better defence against animal and microbial pests, such as against nematodes, insects, mites, phytopathogenic fungi, bacteria, viruses and/or viroids.

In addition to the plants and plant cultivars mentioned above, the treatment according to the invention may also be applied to those which are resistant to one or more abiotic stress factors.

Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention, are those plants characterized by enhanced yield characteristics. Increased yield in said plants can be the result of for example, improved plant physiology, growth and development, such as water use efficiency, water retention efficiency, improved nitrogen use, enhanced carbon assimilation, improved photosynthesis, increased germination efficiency and accelerated maturation. Yield can furthermore by affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or the hybrid effect which results in generally higher yield, vigour, health and resistance towards biotic and abiotic stress factors. Such plants are typically made by crossing an inbred male sterile parent line (the female parent) with another inbred male fertile parent line (the male parent). Hybrid seed is typically harvested from the male sterile plants and sold to growers. Male sterile plants can sometimes (e.g. in corn) be produced by detasseling, (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance. Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide-resistant plants are plants that are made tolerant to acetolactate synthase (ALS) inhibitors. Known ALS-inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in corn events MON863 or MON88017, or the Cry3A protein in maize event MIR604;
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1a and VIP2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG encoding genes of the plants or plants cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as, for example:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes,
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fibre-selective β-1,3-glucanase;
f) plants, such as cotton plants, which have fibres with altered reactivity, for example through the expression of the N-acetylglucosaminetransferase gene including nodC and chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins, are the following which are sold under the trade names YIELD GARD® (for example maize, cotton, soya beans), KnockOut® (for example maize), BiteGard® (for example maize), Bt-Xtra® (for example maize), StarLinls® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, that are listed for example in the databases for various national or regional regulatory agencies (see for example http://gmoinfo.jrc.it/gmp_browse.aspx and http://www.agbios.com/dbase.php).

According to the invention, the plants listed can be treated particularly advantageously with the compounds of the general formula I or the active compound mixtures according to the invention. The preferred ranges indicated above for the active compounds and mixtures also apply to the treatment of these plants. Particular emphasis is given to treating the plants with the compounds and mixtures specifically indicated in the present text.

The active compounds/active compound combinations according to the invention are not only active against plant pests, hygiene pests and stored-product pests, but also, in the sector of veterinary medicine, against animal parasites (ectoparasites and endoparasites) such as hard ticks, soft ticks, scab mites, harvest mites, flies (stinging and licking), parasitic fly larvae, lice, hair lice, bird lice and fleas. These parasites include:

From the order of the Anoplurida, for example Haematopinus spp., Linognathus spp., Pediculus spp., Phtirus spp., Solenopotes spp.

From the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example Pulex spp., Ctenocephalides spp., Xenopsylla spp., Ceratophyllus spp.

From the order of the Heteropterida, for example Cimex spp., Triatoma spp., Rhodnius spp., Panstrongylus spp.

From the order of the Blattarida, for example Blatta orientalis, Periplaneta americana, Blattela germanica, Supella spp.

From the subclass of the Acari (Acarida) and the orders of the Meta- and Mesostigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I)/active compound combinations according to the invention are also suitable for controlling arthropods which attack agricultural livestock such as, for example, cattle, sheep, goats, horses, pigs, donkeys, camels, buffaloes, rabbits, chickens, turkeys, ducks, geese, honeybees, other domestic animals such as, for example, dogs, cats, caged birds, aquarium fish and what are known as experimental animals such as, for example, hamsters, guinea pigs, rats and mice. By controlling these arthropods, it is intended to reduce deaths and performance reductions (in the case of meat, milk, wool, hides, eggs, honey and the like), so that more economical and simpler animal keeping is made possible by the use of the active compounds according to the invention.

In the veterinary sector and in animal keeping, the active compounds/active compound combinations according to the invention are applied in the known manner by enteral administration in the form of, for example, tablets, capsules, drinks, drenches, granules, pastes, boluses, the feed-through method, suppositories, by parenteral administration, such as, for example, by injections (intramuscular, subcutaneous, intravenous, intraperitoneal and the like), implants, by nasal application, by dermal application in the form of, for example, bathing or dipping, spraying, pouring-on and spotting-on, washing, dusting, and with the aid of active-substance-comprising shaped articles such as collars, ear tags, tail tags, limb bands, halters, marking devices and the like.

When used for livestock, poultry, domestic animals and the like, the active substances of the formula (I) can be applied as formulations (for example powders, emulsions, flowables) which comprise the active compounds in an amount of from 1 to 80% by weight, either directly or after 100- to 10 000-fold dilution, or else as a chemical bath.

Moreover, it has been found that the compounds/active compound combinations according to the invention demonstrate a potent insecticidal activity against insects which destroy industrial materials.

The following insects may be mentioned by way of example and by preference, but not by limitation:

beetles such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec., Dinoderus minutus;
hymenoptera such as Sirex juvencus, Urocerus gigas, Urocerus gigas taignus, Urocerus augur;
termites such as Kalotermes flavicollis, Cryptotermes brevis, Heterotermes indicola, Reticulitermes flavipes, Reticulitermes santonensis, Reticulitermes lucifugus, Mastotermes darwiniensis, Zooteimopsis nevadensis, Coptotermes formosanus;
bristletails such as Lepisma saccharina.

Industrial materials are understood as meaning, in the present context, non-live materials such as, preferably, polymers, adhesives, glues, paper and board, leather, timber, derived timber products and paints.

The material to be protected from infestation with insects is very especially preferably timber and derived timber products.

Timber and derived timber products which can be protected by the active compound according to the invention or mixtures containing them are to be understood as meaning by way of example:

structural timber, wooden beams, railway sleepers, components of bridges, jetties, vehicles made of wood, boxes, pallets, containers, telegraph poles, wooden lagging, windows and doors made of wood, plywood, chipboard, joinery or wooden products which are used, quite generally, for building houses or in building joinery.

The active compounds can be used as such, in the form of concentrates or generally customary formulations such as powder, granules, solutions, suspensions, emulsions or pastes.

The formulations mentioned can be prepared in a manner known per se, for example by mixing the active compounds with at least one solvent, diluent, emulsifier, dispersant and/or binder or fixative, water repellent, optionally desiccants and UV stabilizers and, if appropriate, colorants and pigments as well as further processing aids.

The insecticidal compositions or concentrates which are used for the protection of timber and derived timber products comprise the active compound according to the invention in a concentration of from 0.0001 to 95% by weight, in particular from 0.001 to 60% by weight.

The amount of the compositions or concentrates employed depends on the species and the abundance of the insects and on the medium. Upon use, the optimal application rate can be determined in each case by a test series. However, in general it will suffice to employ from 0.0001 to 20% by weight, preferably from 0.001 to 10% by weight, of the active compound, based on the material to be protected.

A suitable solvent and/or diluent is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents with an evaporation number of above 35 and a flashpoint of above 30° C., preferably above 45° C. Such oily and oil-type solvents which are insoluble in water and of low volatility and which are used are suitable mineral oils or their aromatic fractions or mineral-oil-containing solvent mixtures, preferably white spirit, petroleum and/or alkylbenzene.

Mineral oils with a boiling range of 170 to 220° C., white spirit with a boiling range of 170 to 220° C., spindle oil with a boiling range of 250 to 350° C., petroleum and aromatics with a boiling range of 160 to 280° C., oil of turpentine, and the like are advantageously used.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility and with an evaporation number of above 35 and a flashpoint of above 30° C., preferably above 45° C., can be replaced in part by organochemical solvents of high or medium volatility, with the proviso that the solvent mixture also has an evaporation number of above 35 and a flashpoint of above 30° C., preferably above 45° C., and that the mixture is soluble or emulsifiable in this solvent mixture.

In a preferred embodiment, some of the organochemical solvent or solvent mixture is replaced by an aliphatic polar organochemical solvent or solvent mixture. Aliphatic organochemical solvents which contain hydroxyl and/or ester and/or ether groups are preferably used, such as, for example, glycol ethers, esters or the like.

Organochemical binders used for the purposes of the present invention are the synthetic resins and/or binding drying oils which are known per se and which can be diluted in water and/or dissolved or dispersed or emulsified in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin such as indene/coumarone resin, silicone resin, drying vegetable oils and/or drying oils and/or physically drying binders based on a natural and/or synthetic resin.

The synthetic resin employed as binder can be employed in the form of an emulsion, dispersion or solution. Bitumen or bituminous substances may also be used as binders, in amounts of up to 10% by weight. In addition, colorants, pigments, water repellents, odour-masking agents, and inhibitors or anticorrosive agents and the like, all of which are known per se, can be employed.

In accordance with the invention, the material or the concentrate preferably comprises, as organochemical binders, at least one alkyd resin or modified alkyd resin and/or a drying vegetable oil. Alkyd resins which are preferably used in accordance with the invention are those with an oil content of over 45% by weight, preferably 50 to 68% by weight.

Some or all of the abovementioned binder can be replaced by a fixative (mixture) or plasticizer (mixture). These additives are intended to prevent volatilization of the active compounds, and also crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, phosphoric esters such as tributyl phosphate, adipic esters such as di(2-ethylhexyl)adipate, stearates such as butyl stearate or amyl stearate, oleates such as butyl oleate, glycerol ethers or higher-molecular-weight glycol ethers, glycerol esters and p-toluenesulphonic esters.

Fixatives are based chemically on polyvinyl alkyl ethers such as, for example, polyvinyl methyl ether, or ketones such as benzophenone and ethylenebenzophenone.

Other suitable solvents or diluents are, in particular, water, if appropriate as a mixture with one or more of the abovementioned organochemical solvents or diluents, emulsifiers and dispersants.

Particularly effective timber protection is achieved by industrial-scale impregnating processes, for example the vacuum, double-vacuum or pressure processes.

The ready-to-use materials may, if appropriate, comprise further insecticides and, if appropriate, also one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxid, triflumuron, clothianidin, spinosad, tefluthrin,

and also fungicides, such as epoxiconazole, hexaconazole, azaconazole, propiconazole, tebuconazole, cyproconazole, metconazole, imazalil, dichlorfluanid, tolylfluanid, 3-iodo-2-propynyl butylcarbamate, N-octylisothiazolin-3-one and 4,5-dichloro-N-octylisothiazolin-3-one.

The compounds according to the invention can equally be employed for protecting objects which come into contact with saltwater or brackish water, such as hulls, screens, nets, buildings, quaysides and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent stops in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulphide, antimony oxide, polymeric butyl titanate, phenyl(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylenebisthiocarbamate, zinc oxide, copper(I) ethylenebisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as
2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
fungicides such as
benzo[b]thiophenecarboxylic acid cyclohexylamide S,S-dioxide, dichlofluanid, fluorfolpet, 3-iodo-2-propynyl butylcarbamate, tolylfluanid and azoles such as
azaconazole, cyproconazole, epoxiconazole, hexaconazole, metconazole, propiconazole and tebuconazole;
molluscicides such as
fentin acetate, metaldehyde, methiocarb, niclosamid, thiodicarb and trimethacarb, Fe chelates;
or conventional antifouling active compounds such as
4,5-dichloro-2-octyl-4-isothiazolin-3-one, diiodomethylparatiyl sulphone, 2-(N,N-dimethylthio-carbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)pyridine, 2,4,5,6-tetrachloroisophthalonitrile, tetramethylthiuram disulphide and 2,4,6-trichlorophenyl-maleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in saltwater. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguinous, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coloptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The active compounds/active compound combinations according to the invention can also be used as defoliants, desiccants, haulm killers and, in particular, as weed killers. Weeds in the broadest sense are understood as meaning all plants which grow at locations where they are undesired. Whether the substances according to the invention act as nonselective or selective herbicides depends essentially on the application rate.

The active compounds/active compound combinations according to the invention can be used, for example, in the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochioa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds/active compound combinations according to the invention is in no way restricted to these genera, but extends in the same manner to other plants.

Depending on the concentration, the active compounds/active compound combinations according to the invention are suitable for the nonselective weed control on, for example, industrial terrains and railway tracks and on paths and locations with and without trees. Likewise the active compounds according to the invention can be employed for controlling weeds in perennial crops, for example forests, ornamental tree plantings, orchards, vineyards, citrus groves, nut orchards, banana plantations, coffee plantations, tea plantations, rubber plantations, oil palm plantations, cocoa plantations, soft fruit plantations and hop fields, on lawns, turf and pastureland, and for the selective control of weeds in annual crops.

The compounds of the formula (I)/active compound combinations according to the invention have strong herbicidal activity and a broad activity spectrum when used on the soil and on aerial plant parts. To a certain extent, they are also suitable for the selective control of monocotyledonous and dicotyledonous weeds in monocotyledonous and dicotyledonous crops, both pre- and post-emergence.

At certain concentrations or application rates, the active compounds/active compound combinations according to the invention can also be employed for controlling animal pests and fungal or bacterial plant diseases. If appropriate, they can also be used as intermediates or precursors for the synthesis of other active compounds.

The active compounds/active compound combinations can be converted into the customary formulations, such as solutions, emulsions, wettable powders, suspensions, powders, dusting agents, pastes, soluble powders, granules, suspoemulsion concentrates, natural and synthetic materials impregnated with active compound, and very fine capsules in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers.

If the extender used is water, it is also possible to use, for example, organic solvents as auxiliary solvents. Suitable liquid solvents are essentially: aromatics, such as xylene, toluene or alkylnaphthalenes, chlorinated aromatics and chlorinated aliphatic hydrocarbons, such as chlorobenzenes, chloroethylenes or methylene chloride, aliphatic hydrocarbons, such as cyclohexane or paraffins, for example petroleum fractions, mineral and vegetable oils, alcohols, such as butanol or glycol, and also their ethers and esters, ketones, such as acetone, methyl ethyl ketone, methyl isobutyl ketone or cyclohexanone, strongly polar solvents, such as dimethylformamide and dimethyl sulphoxide, and also water.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates, suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian blue, and organic colorants, such as alizarin colorants, azo colorants and metal phthalocyanine colorants, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds/active compound combinations according to the invention, as such or in their formulations, can also be used for weed control purposes as a mixture with known herbicides and/or with substances which improve crop plant tolerance (“safeners”), ready mixes or tank mixes being possible. Mixtures with herbicide products which contain one or more known herbicides and a safener are hence also possible.

A mixture with other known active compounds, such as fungicides, insecticides, acaricides, nematicides, bird repellents, plant nutrients and soil conditioners, is also possible.

The active compounds/active compound combinations can be applied as such, in the form of their formulations or the use forms prepared therefrom by further dilution, such as ready-to-use solutions, suspensions, emulsions, powders, pastes and granules. They are applied in the customary manner, for example by pouring, spraying, atomizing, spreading.

The active compounds/active compound combinations according to the invention can be applied both before and after plant emergence. They can also be incorporated into the soil prior to sowing.

The application rate of active compound can vary within a substantial range. Essentially, it depends on the nature of the desired effect. In general, the application rates are between 1 g and 10 kg of active compound per hectare of soil area, preferably between 5 g and 5 kg per ha.

The advantageous effect of the compatibility with crop plants of the active compound combinations according to the invention is particularly pronounced at certain concentration ratios. However, the weight ratios of the active compounds in the active compound combinations can be varied within relatively wide ranges. In general, from 0.001 to 1000 parts by weight, preferably from 0.01 to 100 parts by weight, particularly preferably 0.05 to 20 parts by weight, of one of the compounds which improves crop plant compatibility (antidotes/safeners) mentioned above under (b) are present per part by weight of active compound of the formula (I).

The active compound combinations according to the invention are generally applied in the form of finished formulations. However, the active compounds contained in the active compound combinations can, as individual formulations, also be mixed during use, i.e. be applied in the form of tank mixes.

For certain application purposes, in particular by the post-emergence method, it may furthermore be advantageous to include, as further additives in the formulations, mineral or vegetable oils which are compatible with plants (for example the commercial preparation “Rako Binol”), or ammonium salts, such as, for example, ammonium sulphate or ammonium thiocyanate.

The novel active compound combinations can be used as such, in the form of their formulations or the use forms prepared therefrom by further dilution, such as ready-to-use solutions, suspensions, emulsions, powders, pastes and granules. Application is in the customary manner, for example by watering, spraying, atomizing, dusting or scattering.

The application rates of the active compound combinations according to the invention can be varied within a certain range; they depend, inter alia, on the weather and on soil factors. In general, the application rates are between 0.001 and 5 kg per ha, preferably between 0.005 and 2 kg per ha, particularly preferably between 0.01 and 0.5 kg per ha.

The active compound combinations according to the invention can be applied before and after emergence of the plants, that is to say by the pre-emergence and post-emergence method.

Depending on their properties, the safeners to be used according to the invention can be used for pretreating the seed of the crop plant (seed dressing) or can be introduced into the seed furrows prior to sowing or be used separately prior to the herbicide or together with the herbicide, before or after emergence of the plants.

In each case, the term “active compounds” or “compounds” also includes the active compound combinations mentioned here.

The preparation and use of the active compounds/active compound combinations according to the invention is illustrated by the examples below.

### PREPARATION EXAMPLES

### Example A.1

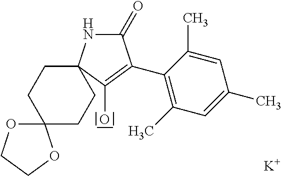

At room temperature, 0.343 g (1 mmol) of the compound (I′-1) are introduced a little at a time into a solution of 10 ml of water and 1 ml of 1 N aqueous potassium hydroxide solution. The mixture is stirred for 1 h and concentrated under reduced pressure on a rotary evaporator, and the residue is precipitated from methyl tert-butyl ether/n-hexane and filtered off with suction.

Yield: 0.36 g (=94% of theory)

Ion chromatography: K⁺ calculated 10.2%

K⁺ found 10.15%

¹H-NMR (400 MHz, d₆-DMSO): δ=1.23, 1.26 (dm, 2H), 1.65-1.76 (m, 4H), 1.90-1.98 (dt, 2H), 2.07 (s, 6H, Ar—CH₃), 2.16 (s, 3H, Ar-4-CH₃), 3.85 (s, 4H, O—CH₂—CH₂—O), 6.63 (s, 2H, Ar—H)

Analogously to Example A.1 and in accordance with the general statements on the preparation of compounds of the formula (I), the following examples are obtained:

(I)

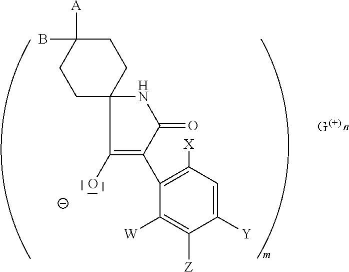

| | | | | | | | | | |
|---|---|---|---|---|---|---|---|---|---|
| Ex.-No. | W | X | Y | Z | A | B | G⁽⁺⁾ⁿ | m | ¹H-NMR (400 MHz, d₆-DMSO) |

| | | | | | | | | |
|---|---|---|---|---|---|---|---|---|
| A.2 | CH₃ | CH₃ | CH₃ | H | —O—(CH₂)₂—O— | Na⁺ | 1 | 1.24, 1.27 (dm, 2H), 1.64-1.77 (m, 4H), 1.91-1.99 |
| | | | | | | | | (dt, 2H), 2.07 (s, 6H, Ar—CH₃), 2.16 (s, 3H, Ar-4-CH₃), 3.85 |
| | | | | | | | | (s, 4H, —O—(CH₂)₂—O), 6.64 (s, 2H, Ar—H) |
| A.3 | CH₃ | CH₃ | CH₃ | H | —O—(CH₂)₂—O— | Li⁺ | 1 | 1.22, 1.26 (dm, 2H), 1.65-1.75 (m, 4H), 1.90-1.98 (dt, |
| | | | | | | | | 2H), 2.07 (s, 6H, Ar—CH₃), 2.16 (s, 3H, Ar-4-CH₃), 3.85 |
| | | | | | | | | (s, 4H, —O—(CH₂)₂—O), 6.63 (s, 2H, Ar—H) |
| A.4 | CH₃ | CH₃ | Cl | H | —O—(CH₂)₂—O— | Li⁺ | 1 | 1.24, 1.26 (dm, 2H), 1.65-1.77 (m, 4H), 1.91-1.99 (dt, |
| | | | | | | | | 2H), 2.12 (s, 6H, Ar—CH₃), 3.85 (s, 4H, O—(CH₂)₂—O), 6.85 |
| | | | | | | | | (s, 2H, Ar—H) |
| A.5 | CH₃ | CH₃ | Cl | H | —O—(CH₂)₂—O— | Na⁺ | 1 | 1.24, 1.27 (dm, 2H), 1.65-1.77 (m, 4H), 1.91-1.98 (dt, 2H), |
| | | | | | | | | 2.12 (s, 6H, Ar—CH₃), 3.85 (s, 4H, O—(CH₂)₂—O), 6.86 |
| | | | | | | | | (s, 2H, Ar—H) |
| A.6 | CH₃ | CH₃ | Cl | H | —O—(CH₂)₂—O— | K⁺ | 1 | 1.23, 1.26 (dm, 2H), 1.64-1.76 (m, 4H), 1.89-1.97 (dt, 2H), |
| | | | | | | | | 2.12 (s, 6H, Ar—CH₃), 3.85 (s, 4H, O—(CH₂)₂—O), 6.84 |
| | | | | | | | | (s, 2H, Ar—H) |
| A.7 | CH₃ | CH₃ | Cl | H | —O—(CH₂)₂—O— | Mg²⁺ | 2 | 1.28, 1.31 (dm, 2H), 1.69-1.72 (m, 4H), 1.93-1.99 (m, 2H), |
| | | | | | | | | 2.12 (s, 6H, Ar—CH₃), 3.86 (s, 4H, O—(CH₂)₂—O), 6.89 |
| | | | | | | | | (s, 2H, Ar—H) |
| A.8 | CH₃ | CH₃ | Cl | H | —O—(CH₂)₂—O— | Ca²⁺ | 2 | 1.25, 1.28 (dm, 2H), 1.66-1.78 (m, 4H), 1.95-1.99 (dt, |
| | | | | | | | | 2H), 2.13 (s, 6H, Ar—CH₃), 3.86 (s, 4H, O—(CH₂)₂—O), 6.87 |
| | | | | | | | | (s, 2H, Ar—H) |

### Preparation of Suspension Concentrates (SC Formulations)

### Comparative Example 1

To prepare a suspension concentrate, initially all liquid components are mixed with one another. In the next step, the solids are added and the mixture is stirred until a homogeneous suspension is formed. The homogeneous suspension is subjected initially to coarse grinding and then to fine grinding, such that a suspension is obtained in which 90% of the solids particles have a particle size of below 10 μm. At room temperature, Kelzan® S and water are then added with stirring until the intended viscosity is reached. This gives a homogeneous suspension concentrate.

| | |
|---|---|
| Compound of the formula A.5 | 25 g |
| Soprophor TS 29 | 40 g |
| Glycerol (99%) | 100 g |
| Citric acid | 1 g |
| Silicone antifoam (Silfoam ® SRE) | 1 g |
| Proxel ® GXL | 1.2 g |
| Preventol ® D 7 | 0.8 g |
| Water | 829.8 g |
| Kelzan ® S | 1.2 g |

### Comparative Example 2

To prepare a suspension concentrate, initially all liquid components are mixed with one another. In the next step, the solids are added and the mixture is stirred until a homogeneous suspension is formed. The homogeneous suspension is subjected initially to coarse grinding and then to fine grinding, such that a suspension is obtained in which 90% of the solids particles have a particle size of below 10 μm. At room temperature, Kelzan® S and water are then added with stirring until the intended viscosity is reached. This gives a homogeneous suspension concentrate. A typical composition of a further SC formulation is:
49 g of the compound of the formula A.5
15 g of Soprophor® TS 54
45 g of Atlox® 4913
100 g of glycerol (99%)
1 g of citric acid
1 g of Silfoam® SRE
1.2 g of Proxel® GXL
0.8 g of Preventol® D7
1.2 g of Kelzan® S
785.8 g of water

### Preparation of Water-Soluble Concentrations (SL Formulations)

To prepare the SL formulation, water, the active compound (I′-2) and urea are initially charged. 2-Molar aqueous sodium hydroxide solution is added until all the material has dissolved, the pH is then adjusted to 10 using 1 molar hydrochloric acid and the mixture is made up with water to 1 l.

A typical composition of an SL formulation was

Compound of the formula

| | |
|---|---|
| A.5 | 50 g |
| Sodium hydroxide solution (2M, aq.) | 51.35 g |
| Hydrochloric acid (1M) | 51.35 g |
| Urea | 102.7 g |
| Preventol ® D 7 | 0.82 g |
| Proxel ® GXL 20% | 1.23 g |
| Silfoam ® SRE | 1.03 g |
| Water | 768.5 g |

Legend:
Soprophor ® TS 54 (tristyrylphenol ethoxylate from Rhodia)
Atlox ® 4913 (acrylic graft copolymer solution from Croda)
Silfoam ® SRE (polydimethylsiloxane)
Kelzan ® S (xanthan gum)
Soprophor TS 29 (tristyrylphenol)

Spray liquor properties of A.5 SL 050 and SC 050 at various pH values

| | | | | Desired amount | | | | | | |
|---|---|---|---|---|---|---|---|---|---|---|
| | | | pH of | of a.i. in 300 l | Weight of | | | | | |
| | Formula- | | the so- | of H₂O | formulation | Assessment of the a.i. behaviour in the spray liquor after | | | | |
| No. | tion type | Solution | lution | [g] | [g] | 0 min | 5 min | 30 min | 1 h | 24 h |
| 1 | SL 050 | 500 ppm | 6.8 | 12 | 0.08 | dissolved | dissolved | dissolved | dissolved | dissolved |
| | | H₂O | | | | | | | | |
| 2 | | buffer | 6.0 | 12 | 0.08 | dissolved | dissolved | dissolved | dissolved | dissolved |
| 3 | | buffer | 8.0 | 12 | 0.08 | dissolved | dissolved | dissolved | dissolved | dissolved |
| 4 | SC 050 | 500 ppm | 5.3 | 12 | 0.08 | not | not | not | not | not |
| | | H₂O | | | | dissolved | dissolved | dissolved | dissolved | dissolved |
| 5 | | buffer | 6.0 | 12 | 0.08 | not | not | not | not | not |
| | | | | | | dissolved | dissolved | dissolved | dissolved | dissolved |
| 6 | | buffer | 8.0 | 12 | 0.08 | not | dissolved | dissolved | dissolved | dissolved |
| | | | | | | dissolved | | | | |
| 7 | SL 050 | 500 ppm | 6.6 | 6 | 0.04 | dissolved | dissolved | dissolved | dissolved | dissolved |
| | | H₂O | | | | | | | | |
| 8 | | buffer | 6.0 | 6 | 0.04 | dissolved | dissolved | dissolved | dissolved | dissolved |
| 9 | | buffer | 8.0 | 6 | 0.04 | dissolved | dissolved | dissolved | dissolved | dissolved |
| 10 | SC 050 | 500 ppm | 5.4 | 6 | 0.04 | not | not | not | not | not |
| | | H₂O | | | | dissolved | dissolved | dissolved | dissolved | dissolved |
| 11 | | buffer | 6.0 | 6 | 0.04 | not | not | almost | almost | almost |
| | | | | | | dissolved | dissolved | dissolved | dissolved | dissolved |
| 12 | | buffer | 8.0 | 6 | 0.04 | not | dissolved | dissolved | dissolved | dissolved |
| | | | | | | dissolved | | | | |

### Use Examples

### Example A

Boosting of penetration into the plant by ammonium salts or phosphonium salts, and synergistic boosting of penetration into the plant by ammonium/phosphonium salts in combination with penetration promoters

This test measured the penetration of active compounds through enzymatically isolated cuticles of apple leaves.

The leaves used were cut in the fully developed state from apple trees of the Golden Delicious variety. The cuticles were isolated as follows:
first of all, leaf discs labelled on the underside with dye and formed by punching were filled by means of vacuum infiltration with a pectinase solution (0.2% to 2% strength) buffered to a pH of between 3 and 4,
sodium azide was then added and
the leaf discs thus treated were left to stand until the original leaf structure broke down and the non-cellular cuticle underwent detachment.

After that, only those cuticles from the top leaf sides that were free from stomata and hairs were used further. They were washed a number of times in alternation with water and with a buffer solution, pH 7. The clean cuticles obtained were, finally, applied to Teflon plaques, smoothed with a gentle jet of air, and dried.

In the next step the cuticular membranes obtained in this way were placed in stainless steel diffusion cells (transport chambers) for the purpose of membrane transport investigations. For these investigations the cuticles were placed centrally using tweezers on the edges of the diffusion cells, which were coated with silicone grease, and sealed with a ring, which was likewise greased. The arrangement had been chosen so that the morphological outer side of the cuticles was directed outwards, in other words facing the air, while the original inner side was facing the inside of the diffusion cell.

The diffusion cells were filled with a 30% strength ethylene glycol/water solution. Penetration was determined by applying 10 μl of the spray liquor of the composition below to the outer side of each of the cuticles. The spray liquor is prepared using local mains water of medium hardness.

After the spray liquors had been applied, the water was evaporated and then the chambers were inverted and placed in thermostated troughs, in which the temperature and humidity over the cuticles was adjustable by means of a gentle stream of air onto the cuticles, with the spray coating (20° C., 60% rh). At regular intervals, samples were taken using an autosampler, and the amount of active compound was determined using HPLC.

The results of the experiment are apparent from the table below. The numbers stated represent average values from 8 to 10 measurements.

TABLE A

| | Penetration after 24 h/% | |
|---|---|---|
| | SC 050 + RME (1 g/l) + | SL 050 + RME (1 g/l) + |
| Active compound | AS (1 g/l) | AS (1 g/l) |
| Example A.5 | 3 | 7 |

RME = rapeseed oil methyl ester (formulated for use as 500 EW, concentration stated in g of active compound/1)
AS = ammonium sulphate

### Example B

### Myzus persicae Test

### MYZUPE Translaminar

To produce a suitable solution of the preparation, the formulation is diluted with water to the desired concentration. If the addition of ammonium salts and penetrant is required, the appropriate amount is pipetted in after dilution of the respective finished solution of the preparation.

Cabbage plants (Brassica oleracea) which are heavily infested by the green peach aphid (Myzus persicae) are treated by spraying the upper side of the leaves with the solution of the preparation of the desired concentration.

After the desired period of time, the kill in % is determined. Here, 100% means that all of the animals have been killed; 0% means that none of the animals have been killed.

In this test, for example, the following compounds of the Preparation Examples show good activity compared to a conventional formulation:

| | Concentration | Kill |
|---|---|---|
| Formulation | in g of ai/ha | in % after 7^{d} |
| A.5 SL 050 + | 3 | 90 |
| RME EW 500 + AMS (1 g ai/l) | | |
| according to the invention | | |
| A.5 SC 050 + | 3 | 40 |
| RME EW 500 + AMS (1 g ai/l) | | |
| prior art | | |

AMS = ammonium sulphate

### Example C-1

In plots of a size of about 8 m², cotton plants of the cultivar “Delta Opal” of a height of about 15 cm are treated in three replications against Aphis gossypii using a pressure-operated knapsack sprayer (3 bar). Here, the active compound A.5 is applied as SC050 and SL050 at the stated application rate in a tank mix of 1 g/l of ammonium sulphate and 1 g/l of a.i. rapeseed oil methyl ester EW 500 using an application rate of 300 l of water/ha. Evaluation is carried out 1 and 3 days after the application by scoring the kill of the nymphs on the leaves.

| | | |
|---|---|---|
| | | Effect Abbott (%) |
| Active compound | Application rate | Aphis gossypii |

| A.5 | g a.i./ha | 1 d | 3 d |
|---|---|---|---|
| SC 050 | 24 | 61.1 | 85.7 |
| SL 050 | 24 | 62.8 | 93.6 |

### Example C-2

In plots of a size of 10 m², cotton plants of the cultivar “Fibermax” are treated in three replications against Aphis gossypii using a pressure-operated knapsack sprayer (2.5 bar). Here, the active compound A.5 is applied as SC050 and SL050 at the stated application rate in a tank mix of 1 g/l of ammonium sulphate and 1 g/l of a.i. rapeseed oil methyl ester EW 500 in comparison to the standard imidacloprid (SC 050) using an application rate of 300 l of water/ha. Evaluation is carried out 3, 6, 9, 13 and 17 days after the application by counting the number of live animals on the leaves. Subsequently, the efficacy in percent is calculated according to Henderson and Tilton.

| | | Efficacy (% H + T) | | | | |
|---|---|---|---|---|---|---|
| Active compound | Application rate | Aphis gossypii | | | | |
| A.5 | g a.i./ha | 3 d | 6 d | 9 d | 13 d | 17 d |
| SC 050 | 24 | 75.8 | 78.2 | 83.1 | 84.1 | 71.6 |
| SC 050 | 12 | 66.6 | 71.3 | 56.1 | 55.9 | 28.0 |
| SL 050 | 12 | 81.4 | 88.0 | 83.1 | 85.2 | 76.1 |
| imidacloprid | 105 | 78.1 | 84.1 | 83.7 | 82.0 | 64.7 |

### Example C-3

In plots of a size of 15 m², cotton plants of the cultivar “Fibermax 977” are treated in three replications against Tetranychus urticae using a pressure-operated knapsack sprayer (4 bar). Here, the active compound A.5 is applied as SC050 and SL050 at the stated application rate in a tank mix of 1 g/l of ammonium sulphate and 1 g/l of a.i. rapeseed oil methyl ester EW 500 and the standard abamectin (EC 018) using an application rate of in each case 300 l of water/ha. Two applications are carried out at an interval of 8 days. Evaluation is carried out 3, 7, 11 and 15 days after the first application by scoring the kill of the spider mites on the leaves.

| | | Effect Abbott (%) | | | |
|---|---|---|---|---|---|
| Active compound | Application rate | Tetranychus urticae | | | |
| A.5 | g a.i./ha | 3 d | 7 d | 11 d | 15 d |
| SC 050 | 12 | 41.7 | 28.2 | 10.3 | 30.7 |
| SL 050 | 12 | 40.0 | 33.9 | 18.7 | 48.3 |
| abamectin | 9 | 49.3 | 27.2 | 5.8 | 40.7 |

### Example D

### Phaedon Test

### Spray Treatment

Solvents: 78.0 parts by weight of acetone
1.5 parts by weight of dimethylformamide

Emulsifier: 0.5 part by weight of alkylaryl polyglycol ether

To produce a suitable preparation of active compound, 1 part by weight of active compound is mixed with the stated amounts of solvents and emulsifier, and the concentrate is diluted with emulsifier-containing water to the desired concentration.

Discs of Chinese cabbage (Brassica pekinensis) are sprayed with an active compound preparation of the desired concentration and, after drying, populated with larvae of the mustard beetle (Phaedon cochleariae).

After the desired period of time, the effect in % is determined. Here, 100% means that all of the beetle larvae have been killed; 0% means that none of the beetle larvae have been killed.

In this test, for example, the following compounds of the Preparation Examples show an activity of ≧80% at an application rate of 500 g/ha:

Ex. Nos. A1, A2, A3, A4, A6, A7, A8

### Example E

### Myzus Test

### MYZUPE Spray Treatment

Solvents: 78.0 parts by weight of acetone
1.5 parts by weight of dimethylformamide

Emulsifier: 0.5 part by weight of alkylaryl polyglycol ether

To produce a suitable preparation of active compound, 1 part by weight of active compound is mixed with the stated amounts of solvents and emulsifier, and the concentrate is diluted with emulsifier-containing water to the desired concentration.

Discs of Chinese cabbage (Brassica pekinensis) which are infested by all stages of the green peach aphid (Myzus persicae) are sprayed with an active compound preparation of the desired concentration.

After the desired period of time, the effect in % is determined. Here, 100% means that all of the aphids have been killed; 0% means that none of the aphids have been killed.

In this test, for example, the following compounds of the Preparation Examples show an activity of ≧80% at an application rate of 500 g/ha:

Ex. Nos. A1, A2, A3, A4, A5, A6, A7, A8

### Example F

### Tetranychus Test, OP-Resistant

### TETRUR Spray Treatment

Solvents: 78.0 parts by weight of acetone
1.5 parts by weight of dimethylformamide

Emulsifier: 0.5 part by weight of alkylaryl polyglycol ether

To produce a suitable preparation of active compound, 1 part by weight of active compound is mixed with the stated amounts of solvents and emulsifier, and the concentrate is diluted with emulsifier-containing water to the desired concentration.

Discs of bean leaves (Phaseolus vulgaris) which are infested by all stages of the greenhouse red spider mite (Tetranychus urticae) are sprayed with an active compound preparation of the desired concentration.

After the desired period of time, the effect in % is determined. Here, 100% means that all of the spider mites have been killed; 0% means that none of the spider mites have been killed.

In this test, for example, the following compounds of the Preparation Examples show an activity of ≧80% at an application rate of 100 g/ha:

Ex. Nos. A2, A5, A6

### Example G

### Myzus persicae Test

### MYZUPE

Solvent: 7 parts by weight of dimethylformamide

Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration. If the addition of ammonium salts, penetrant or ammonium salts and penetrant is required, these are pipetted in at a concentration of 1000 ppm after dilution of the respective finished solution of the preparation.

Bell pepper plants (Capsicum annuum) which are heavily infested by the green peach aphid (Myzus persicae) are treated by spraying with the active compound preparation of the desired concentration.

After the desired period of time, the kill in % is determined. Here, 100% means that all of the animals have been killed; 0% means that none of the animals have been killed.

In this test, for example, the following compounds of the Preparation Examples show good activity: see Table

| | | Active | |
|---|---|---|---|
| Active | Animal | compound conc. | |
| compound | species | ppm | % effect after 7 d |
| Ex. I-1-a-2 | MYZUPE | 0.8 | 50 |
| known from | | | |
| WO 06/089633 | | | |
| A.2 | MYZUPE | 0.8 | 90 |
| according to the | | | |
| invention | | | |
| A.1 | MYZUPE | 0.8 | 75 |
| according to the | | | |
| invention | | | |
| A.3 | MYZUPE | 0.8 | 75 |
| according to the | | | |
| invention | | | |
| Ex. I-1-a-4 | MYZUPE | 10 | 75 |
| known from | | | |
| WO 06/089633 | | | |
| A.6 | MYZUPE | 10 | 95 |
| according to the | | | |
| invention | | | |
| A.4 | MYZUPE | 10 | 100 |
| according to the | | | |
| invention | | | |
| A.7 | MYZUPE | 10 | 98 |
| according to the | | | |
| invention | | | |

### Example H

### Aphis gossypii Test

### APHIGO Spray Treatment

Solvent: 7 parts by weight of dimethylformamide

Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration. If the addition of ammonium salts, penetrant or ammonium salts and penetrant is required, these are pipetted in at a concentration of 1000 ppm after dilution of the respective finished solution of the preparation.

Cotton leaves (Gossypium hirsutum) which are heavily infested by the cotton aphid (Aphis gossypii) are sprayed with an active compound preparation of the desired concentration.

After the desired period of time, the kill in % is determined. Here, 100% means that all of the aphids have been killed; 0% means that none of the aphids have been killed.

In this test, for example, the following compounds of the Preparation Examples show good activity: see Table

| | | Active | |
|---|---|---|---|
| Active | Animal | compound conc. | |
| compound | species | ppm | % effect after 7 d |
| Ex. I-1-a-2 | APHIGO | 0.8 | 55 |
| known from | | | |
| WO 06/089633 | | | |
| A.3 | APHIGO | 0.8 | 75 |
| according to the | | | |
| invention | | | |

### Example I

### Tetranychus Test

### OP-Resistant

Solvent: 7 parts by weight of dimethylformamide

Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration. If the addition of ammonium salts, penetrant or ammonium salts and penetrant is required, these are pipetted in at a concentration of 1000 ppm after dilution of the respective finished solution of the preparation.

Bean plants (Phaseolus vulgaris) which are heavily infested by all stages of the greenhouse red spider mite (Tetranychus urticae) are treated by spraying with the active compound preparation of the desired concentration.

After the desired period of time, the effect in % is determined. Here, 100% means that all of the spider mites have been killed; 0% means that none of the spider mites have been killed.

In this test, for example, the following compounds of the Preparation Examples show good activity: see Table

| | | Active | |
|---|---|---|---|
| Active | Animal | compound conc. | |
| compound | species | ppm | % effect after 7 d |
| Ex. I-1-a-2 | TETRUR | 4 | 50 |
| known from | | | |
| WO 06/089633 | | | |
| A.2 | TETRUR | 4 | 70 |
| according to the | | | |
| invention | | | |

1. A compound of formula (I) 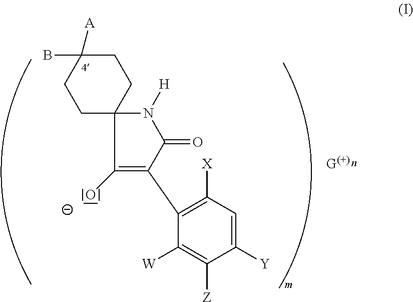
in which:
W represents hydrogen, alkyl, alkenyl, alkynyl, halogen, alkoxy, haloalkyl, haloalkoxy or cyano,
X represents halogen, alkyl, alkenyl, alkynyl, alkoxy, haloalkyl, haloalkoxy, nitro or cyano,
Y and Z independently of one another represent hydrogen, alkyl, alkenyl, alkynyl, alkoxy, halogen, haloalkyl, haloalkoxy, cyano or nitro,
A and B and the carbon atom to which they are attached represent a five- to seven-membered ketal which is in each case optionally substituted by alkyl, haloalkyl, alkoxy, alkoxyalkyl or optionally substituted phenyl,
G represents a metal ion equivalent or ammonium ion,
m represents the number 1 or 2,
n represents the number 1 or 2.

2. The compound of formula (I) according to claim 1
in which:
W represents hydrogen, chlorine, bromine, C₁-C₄-alkyl, C₁-C₄-alkoxy, C₁-C₂-haloalkyl or C₁-C₂-haloalkoxy,
X represents chlorine, bromine, C₁-C₄-alkyl, C₁-C₄-alkoxy, C₁-C₄-haloalkyl, C₁-C₄-haloalkoxy or cyano,
Y and Z independently of one another represent hydrogen, fluorine, chlorine, bromine, C₁-C₄-alkyl, C₁-C₆-alkoxy, C₁-C₄-haloalkyl, C₁-C₄-haloalkoxy or cyano,
A and B and the carbon atom to which they are attached represent a five- or six-membered ketal which is optionally mono- to trisubstituted by C₁-C₄-alkyl, C₁-C₃-haloalkyl, C₁-C₄-alkoxy or C₁-C₄-alkoxy-C₁-C₂-alkyl,
G represents lithium, sodium, potassium, caesium, magnesium-halogen cations, magnesium, calcium or an ammonium ion 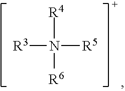
in which R³, R⁴, R⁵, R⁶ independently of one another represent hydrogen, C₁-C₈ alkyl, C₁-C₄-alkoxy-C₂-C₄-alkyl, poly-(C₁-C₄-alkoxy)-C₂-C₄-alkyl, C₃-C₈-alkenyl, C₁-C₈-alkoxy or optionally halogen-, alkyl- or alkoxy-substituted benzyl,
m represents the number 1 or 2,
n represents the number 1 or 2.

3. The compound of formula (I) according to claim 1 in which:
W represents hydrogen, chlorine, bromine, methyl, ethyl, methoxy, ethoxy or trifluoromethyl,
X represents chlorine, bromine, methyl, ethyl, methoxy, ethoxy, trifluoromethyl, difluoromethoxy, trifluoromethoxy or cyano,
Y and Z independently of one another represent hydrogen, fluorine, chlorine, bromine, methyl, ethyl, methoxy, trifluoromethyl, trifluoromethoxy or cyano,
A and B and the carbon atom to which they are attached represent a five- or six-membered ketal which is optionally mono- or disubstituted by methyl, ethyl, propyl, trifluoromethyl, monochloromethyl, methoxy, ethoxy, methoxymethyl or ethoxymethyl,
G represents lithium, sodium, potassium, caesium, a magnesium chloride cation, a magnesium bromide cation, a magnesium iodide cation, magnesium, calcium or an ammonium ion 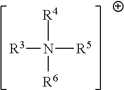
in which:
R³, R⁴, R⁵, R⁶ independently of one another represent hydrogen, C₁-C₆-alkyl or benzyl,
m represents the number 1 or 2,
n represents the number 1 or 2.

4. The compound of formula (I) according to claim 1 in which:
W represents hydrogen, chlorine, bromine, methyl, ethyl or methoxy,
X represents chlorine, bromine, methyl, ethyl, methoxy or ethoxy,
Y and Z independently of one another represent hydrogen, chlorine, bromine or methyl,
A and B and the carbon atom to which they are attached represent a five- or six-membered ketal which is optionally mono- or disubstituted by methyl, ethyl, propyl, monochloromethyl or methoxymethyl,
G represents lithium, sodium, potassium, caesium, a magnesium bromide cation, magnesium, calcium or an ammonium ion 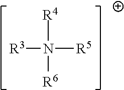
in which:
R³, R⁴, R⁵, R⁶ independently of one another represent C₁-C₄-alkyl or benzyl,
m represents the number 1 or 2,
n represents the number 1 or 2.

5. A process for preparing a compound of formula (I) according to claim 1, comprising,
(A)
intramolecularly condensing a compound of formula (II) 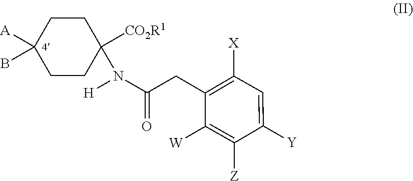
in which:
A, B, W, X, Y and Z have the meanings given above,
and
R¹ represents alkyl,
in the presence of a diluent and a metal base, or
(B)
reacting a compound of formula (I′), 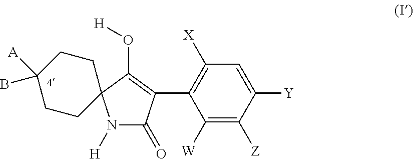
in which A, B, W, X, Y and Z have the meanings given above,
a) with a metal compound of formula (III) or (IV)
G(OR²)ₙ (III), G(H)ₙ (IV)
in which:
G represents a mono- or divalent metal,
n represents the number 1 or 2 and
R² represents hydrogen or alkyl,
optionally in the presence of a diluent
or
b) with an amine of formula (V) or an ammonium compound of formula (VI) 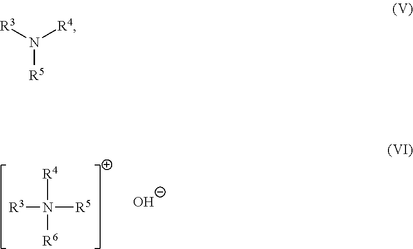
in which:
R³, R⁴, R⁵, R⁶ independently of one another represent hydrogen, C₁-C₈ alkyl, C₁-C₄-alkoxy-C₂-C₄-alkyl, poly-(C₁-C₄-alkoxy)-C₂-C₄-alkyl, C₃-C₈-alkenyl, C₁-C₈-alkoxy or optionally halogen-, alkyl- or alkoxy-substituted benzyl, optionally
in the presence of a diluent.

6. A composition for controlling pests and/or unwanted plant growth, comprising at least one compound of formula (I) according to claim 1.

7. A method for controlling animal pests and/or unwanted plant growth, comprising applying to said pests, unwanted plant growth or their habitat at least one compound of formula (I) according to claim 1.

8. (canceled)

9. A process for preparing a composition for controlling pests and/or unwanted plant growth, comprising mixing at least one compound of formula (I) according to claim 1 with extenders, surfactants or a combination thereof.

10. (canceled)

11. A composition comprising an effective amount of an active compound combination comprising,
(a′) at least one compound of formula (I) according to claim 1,
and
(b′) at least one crop plant compatibility-improving compound:
4-dichloroacetyl-1-oxa-4-azaspiro[4.5]decane 1-dichloroacetylhexahydro-3,3,8a-trimethylpyrrolo[1,2-a]pyrimidin-6(2H)-one (dicyclonon), 4-dichloroacetyl-3,4-dihydro-3-methyl-2H-1,4-benzoxazine (benoxacor), 1-methylhexyl 5-chloroquinoline-8-oxyacetate (cloquintocet-mexyl), 3-(2-chlorobenzyl)-1-(1-methyl-1-phenylethyl)urea (cumyluron), α-(cyanomethoximino)phenylacetonitrile (cyometrinil), 2,4-dichlorophenoxyacetic acid, 4-(2,4-dichlorophenoxy)butyric acid, 1-(1-methyl-1-phenylethyl)-3-(4-methylphenyl)urea (daimuron, dymron), 3,6-dichloro-2-methoxybenzoic acid (dicamba), S-1-methyl-1-phenylethyl piperidine-1-thiocarboxylate (dimepiperate), 2,2-dichloro-N-(2-oxo-2-(2-propenylamino)ethyl)-N-(2-propenyl)acetamide, 2,2-dichloro-N,N-di-2-propenylacetamide (dichlormid), 4,6-dichloro-2-phenylpyrimidine (fenclorim), ethyl 1-(2,4-dichlorophenyl)-5-trichloromethyl-1H-1,2,4-triazole-3-carboxylate (fenchlorazole-ethyl), phenylmethyl 2-chloro-4-trifluoromethylthiazole-5-carboxylate (flurazole), 4-chloro-N-(1,3-dioxolan-2-ylmethoxy)-α-trifluoroacetophenone oxime (fluxofenim), 3-dichloroacetyl-5-(2-furanyl)-2,2-dimethyloxazolidine (furilazole), ethyl 4,5-dihydro-5,5-diphenyl-3-isoxazolecarboxylate (isoxadifen-ethyl), 1-(ethoxycarbonyl)ethyl 3,6-dichloro-2-methoxybenzoate (lactidichlor), (4-chloro-o-tolyloxy)acetic acid, 2-(4-chloro-o-tolyloxy)propionic acid (mecoprop), diethyl 1-(2,4-dichorophenyl)-4,5-dihydro-5-methyl-1H-pyrazole-3,5-dicarboxylate (mefenpyr-diethyl), 2-dichloromethyl-2-methyl-1,3-dioxolane, 2-propenyl-1-oxa-4-azaspiro[4.5]decane-4-carbodithioate, 1,8-naphthalic anhydride, α-(1,3-dioxolan-2-ylmethoximino)phenylacetonitrile (oxabetrinil), 2,2-dichloro-N-(1,3-dioxolan-2-ylmethyl)-N-(2-propenyl)acetamide, 3-dichloroacetyl-2,2-dimethyloxazolidine, 3-dichloroacetyl-2,2,5-trimethyloxazolidine, 4-(4-chloro-o-tolyl)butyric acid, 4-(4-chlorophenoxy)butyric acid, diphenylmethoxyacetic acid, methyl diphenylmethoxyacetate, ethyl diphenylmethoxyacetate, methyl 1-(2-chlorophenyl)-5-phenyl-1H-pyrazole-3-carboxylate, ethyl 1-(2,4-dichlorophenyl)-5-methyl-1H-pyrazole-3-carboxylate, ethyl 1-(2,4-dichlorophenyl)-5-isopropyl-1H-pyrazole-3-carboxylate, ethyl 1-(2,4-dichlorophenyl)-5-(1,1-dimethylethyl)-1H-pyrazole-3-carboxylate, ethyl 1-(2,4-dichlorophenyl)-5-phenyl-1H-pyrazole-3-carboxylate, ethyl 5-(2,4-dichlorobenzyl)-2-isoxazoline-3-carboxylate, ethyl 5-phenyl-2-isoxazoline-3-carboxylate, ethyl 5-(4-fluorophenyl)-5-phenyl-2-isoxazoline-3-carboxylate, 1,3-dimethylbut-1-yl 5-chloroquinoline-8-oxyacetate, 4-allyloxybutyl 5-chloroquinoline-8-oxyacetate, 1-allyloxyprop-2-yl 5-chloroquinoline-8-oxyacetate, methyl 5-chloroquinoxaline-8-oxyacetate, ethyl 5-chloroquinoline-8-oxyacetate, allyl 5-chloroquinoxaline-8-oxyacetate, 2-oxoprop-1-yl 5-chloroquinoline-8-oxyacetate, diethyl 5-chloroquinoline-8-oxymalonate, diallyl 5-chloroquinoxaline-8-oxymalonate, diethyl 5-chloroquinoline-8-oxymalonate, 4-carboxychroman-4-ylacetic acid, 4-chlorophenoxyacetic acid, 3,3′-dimethyl-4-methoxybenzophenone, 1-bromo-4-chloromethylsulphonylbenzene, 1-[4-(N-2-methoxybenzoylsulphamoyl)phenyl]-3-methylurea, 1-[4-(N-2-methoxybenzoylsulphamoyl)phenyl]-3,3-dimethylurea, 1-[4-(N-4,5-dimethylbenzoylsulphamoyl)phenyl]-3-methylurea, 1-[4-(N-naphthylsulphamoyl)phenyl]-3,3-dimethylurea, or N-(2-methoxy-5-methylbenzoyl)-4-(cyclopropylaminocarbonyl)benzenesulphonamide,
or a compound of formula (IIa) 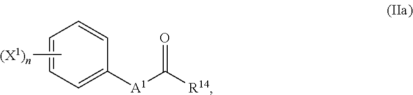
or of the general formula (IIb) 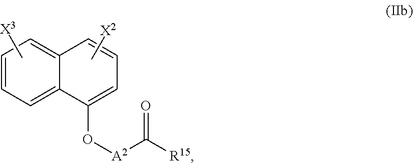
or of formula (IIc) 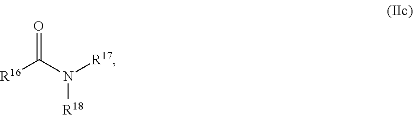
where
n represents a number from 0 and 5,
A¹ represents one of the divalent heterocyclic groups shown below, 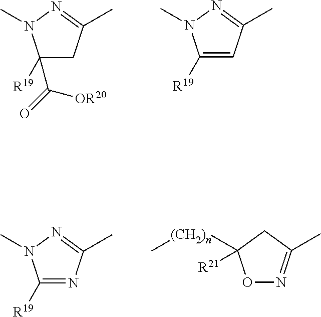
A² represents optionally C₁-C₄-alkyl- and/or C₁-C₄-alkoxycarbonyl-substituted alkanediyl having 1 or 2 carbon atoms,
R¹⁴ represents hydroxyl, mercapto, amino, C₁-C₆-alkoxy, C₁-C₆-alkylthio, C₁-C₆-alkylamino or di(C₁-C₄-alkyl)amino,
R¹⁵ represents hydroxyl, mercapto, amino, C₁-C₆-alkoxy, C₁-C₆-alkylthio, C₁-C₆-alkylamino or di(C₁-C₄-alkyl)amino,
R¹⁶ represents in each case optionally fluorine-, chlorine- and/or bromine-substituted C₁-C₄-alkyl,
R¹⁷ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-alkyl-substituted phenyl,
R¹⁸ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-alkyl-substituted phenyl, or together with R¹⁷ represents C₃-C₆-alkanediyl or C₂-C₅-oxalkanediyl, each of which is optionally substituted by C₁-C₄-alkyl, phenyl, furyl, a fused benzene ring or by two substituents which, together with the C atom to which they are attached, form a 5- or 6-membered carbocycle,
R¹⁹ represents hydrogen, cyano, halogen, or represents in each case optionally fluorine-, chlorine- and/or bromine-substituted C₁-C₄-alkyl, C₃-C₆-cycloalkyl or phenyl,
R²⁰ represents hydrogen, in each case optionally hydroxyl-, cyano-, halogen- or C₁-C₄-alkoxy-substituted C₁-C₆-alkyl, C₃-C₆-cycloalkyl or tri(C₁-C₄-alkyl)silyl,
R²¹ represents hydrogen, cyano, halogen, or represents in each case optionally fluorine-, chlorine- and/or bromine-substituted C₁-C₄-alkyl, C₃-C₆-cycloalkyl or phenyl,
X¹ represents nitro, cyano, halogen, C₁-C₄-alkyl, C₁-C₄-haloalkyl, C₁-C₄-alkoxy or C₁-C₄-haloalkoxy,
X² represents hydrogen, cyano, nitro, halogen, C₁-C₄-alkyl, C₁-C₄-haloalkyl, C₁-C₄-alkoxy or C₁-C₄-haloalkoxy,
X³ represents hydrogen, cyano, nitro, halogen, C₁-C₄-alkyl, C₁-C₄-haloalkyl, C₁-C₄-alkoxy or C₁-C₄-haloalkoxy,
or a compound of formula (IId) 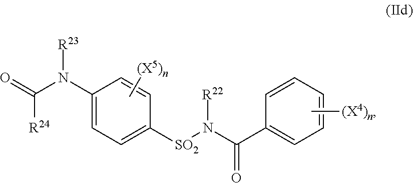
or of formula (IIe) 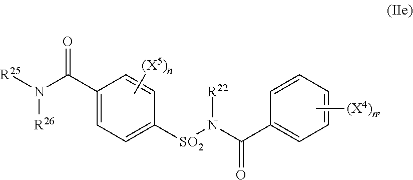
where
n represents a number from 0 to 5,
R²² represents hydrogen or C₁-C₄-alkyl,
R²³ represents hydrogen or C₁-C₄-alkyl,
R²⁴ represents hydrogen, in each case optionally cyano-, halogen- or C₁-C₄-alkoxy-substituted C₁-C₆-alkyl, C₁-C₆-alkoxy, C₁-C₆-alkylthio, C₁-C₆-alkylamino or di(C₁-C₄-alkyl)amino, or in each case optionally cyano-, halogen- or C₁-C₄-alkyl-substituted C₃-C₆-cycloalkyl, C₃-C₆-cycloalkyloxy, C₃-C₆-cycloalkylthio or C₃-C₆-cycloalkylamino,
R²⁵ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, or optionally cyano-, halogen- or C₁-C₄-alkyl-substituted C₃-C₆-cycloalkyl,
R²⁶ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, optionally cyano-, halogen- or C₁-C₄-alkyl-substituted C₃-C₆-cycloalkyl, or optionally nitro-, cyano-, halogen-, C₁-C₄-alkyl-, C₁-C₄-haloalkyl-, C₁-C₄-alkoxy- or C₁-C₄-haloalkoxy-substituted phenyl, or together with R²⁵ represents in each case optionally C₁-C₄-alkyl-substituted C₂-C₆-alkanediyl or C₂-C₅-oxalkanediyl,
X⁴ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy, and
X⁵ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy.

12. The composition according to claim 11 where the crop plant compatibility-improving compound is selected from the group consisting of compounds:
cloquintocet-mexyl, fenchlorazole-ethyl, isoxadifen-ethyl, mefenpyr-diethyl, furilazole, fenclorim, cumyluron, dymron,

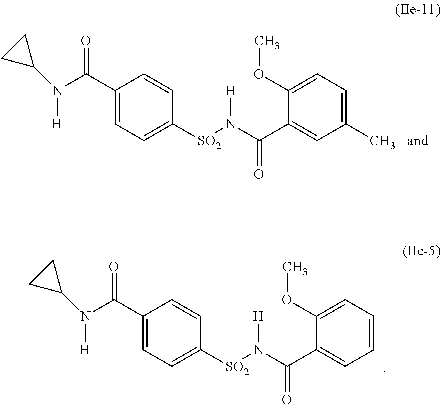

13. The composition according to claim 11 or 12 where the crop plant compatibility-improving compound is cloquintocet-mexyl.

14. The composition according to claim 11 or 12 where the crop plant compatibility-improving compound is mefenpyr-diethyl.

15. A composition comprising a phase which comprises at least one dissolved compound of formula (I) according to claim 1, or a compound of formula (I′) 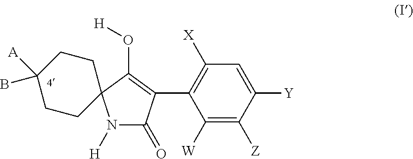
in which A, B, W, X, Y, and Z have the meanings given above,
and at least one solvent.

16. The composition according to claim 15, wherein the solvent is water.

17. A composition comprising
at least one compound of formula (I) according to claim 1 or a composition according to claim 11, and
at least one salt of formula (II′) 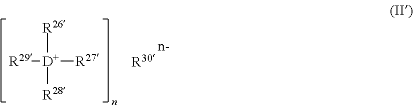
in which:
D represents nitrogen or phosphorus,
R^{26′}, R^{27′}, R^{28′} and R^{29′} independently of one another represent hydrogen or in each case optionally substituted C₁-C₈-alkyl or mono- or polyunsaturated, optionally substituted C₁-C₈-alkylene, the substituents are halogen, nitro or cyano,
n represents 1, 2, 3 or 4,
R^{30′} represents an organic or inorganic anion.

18. The composition according to claim 17, further comprises at least one penetrant.

19. A method of increasing the pesticidal and/or herbicidal activity of a compound of formula (I) according to claim 1 or a composition according to claim 11, comprising preparing a ready-to-use spray liquor composition using a salt of formula (II′) 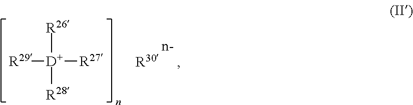
in which
D, R^{26′}, R^{27′}, R^{28′} and R^{29′} have the meaning given above.

20. The method according to claim 19, where the ready-to-use composition further comprises a penetrant.

21. A method for controlling pests and/or unwanted plant growth, comprising applying an effective amount of a composition according to claim 11 or a composition according to claim 15 to the pests, unwanted plant growth, or their habitat.

22. (canceled)

23. A process for preparing a composition for controlling pests and/or unwanted plant growth, comprising mixing a composition according to claim 15 with extenders, surfactants or a combination thereof.

24. (canceled)

25. A process for preparing a composition according to claim 15, comprising adding all required components of the composition to a water-miscible solvent or water.

26. A method for controlling unwanted plant growth and/or pests, comprising applying at least one compound of formula (I) according to claim 1 and at least one crop plant compatibility-improving compound according to claim 11 separately in close temporal succession to the plants, pests or their surroundings.

27. A compound of formula A.1, A.2, A.3, A.4, A.5, A.6, A.7, or A.8:

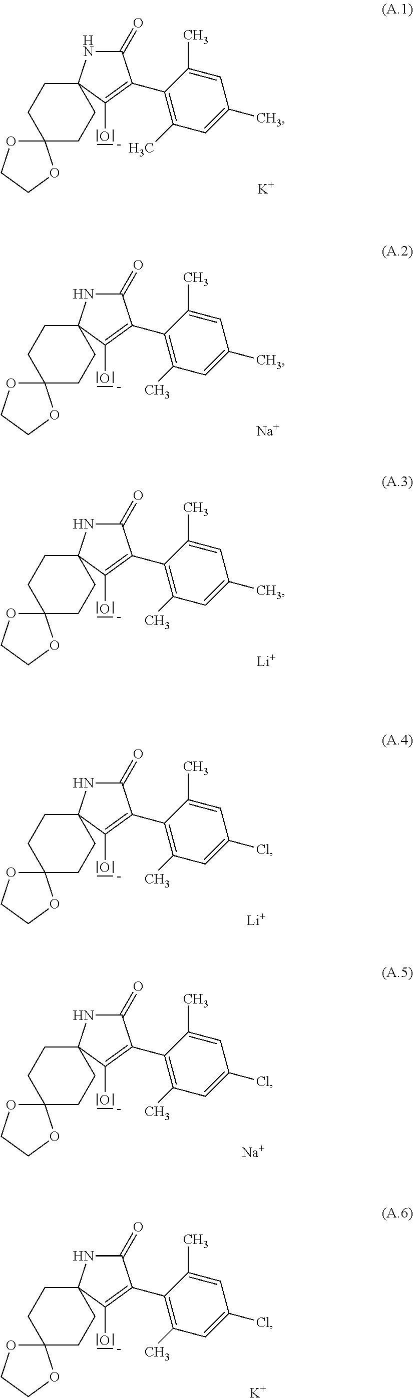

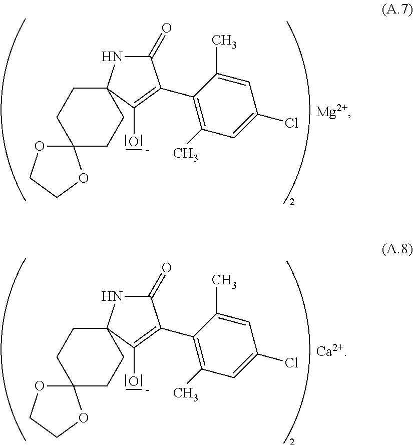

28. A method for controlling pests and/or unwanted plant growth, comprising applying an effective amount of a composition according to claim 16 to the pests, unwanted plant growth, or their habitat.

29. A method for controlling pests and/or unwanted plant growth, comprising applying an effective amount of a composition according to claim 17 to the pests, unwanted plant growth, or their habitat.

30. A method for controlling pests and/or unwanted plant growth, comprising applying an effective amount of a composition according to claim 18 to the pests, unwanted plant growth, or their habitat.

31. A process for preparing a composition for controlling pests and/or unwanted plant growth, comprising mixing a composition according to claim 16 with extenders, surfactants or a combination thereof.

32. A process for preparing a composition for controlling pests and/or unwanted plant growth, comprising mixing a composition according to claim 17 with extenders, surfactants or a combination thereof.

33. A process for preparing a composition for controlling pests and/or unwanted plant growth, comprising mixing a composition according to claim 18 with extenders, surfactants or a combination thereof.
